(19) United States
(12) Patent Application Publication
(10) Pub. No.: US 2024/0139081 A1
(43) Pub. Date: May 2, 2024

(54) BIODEGRADABLE DELIVERY PARTICLES

(71) Applicant: Encapsys, LLC, Appleton, WI (US)

(72) Inventors: Susana FERNÁNDEZ PRIETO, Brussels (BE); Valerie Francine Hans EYKENS, Brussels (BE); Walter Franciscus Joanna VANDERVEKEN, Brussels (BE); Rita DEL PEZZO, Brussels (BE); Johan SMETS, Brussels (BE); Linsheng FENG, Menasha, WI (US); Fadi Selim CHAKAR, Neenah (BE); Travis Ian BARDSLEY, Appleton, WI (US); Robert Stanley BOBNOCK, Menasha, WI (US)

(73) Assignee: Encapsys, LLC, Appleton, WI (US)

(21) Appl. No.: 18/252,822

(22) PCT Filed: Nov. 18, 2021
(86) PCT No.: PCT/US2021/059864

§ 371 Date: May 12, 2023

Related U.S. Application Data
(60) Provisional application No. 63/116,139, filed on Nov. 19, 2020.

(51) Int. Cl. A61K 8/11; A01N 25/28; A61K 8/81; A61Q 5/02; B01J 13/16; C11D 3/00; C11D 3/22; C11D 3/37; C11D 3/50

(52) U.S. Cl. CPC A61K 8/11; A01N 25/28; A61K 8/8129; A61Q 5/02; B01J 13/16; C11D 3/001; C11D 3/227; C11D 3/3753; C11D 3/505; A61K 2800/56

(57) ABSTRACT

A biodegradable delivery particle having a benefit agent containing core and a shell.

### CROSS REFERENCE TO RELATED APPLICATIONS

Encapsys, LLC (formerly known as the Encapsys division of Appleton Papers Inc.) and The Procter & Gamble Company executed a Joint Research Agreement on or about Nov. 28, 2005 and this invention was made as a result of activities undertaken within the scope of that Joint Research Agreement between the parties that was in effect on or before the date of this invention.

### FIELD OF THE INVENTION

The invention relates to biodegradable delivery particles having a benefit agent containing core and a shell.

### BACKGROUND OF THE INVENTION

Microencapsulation is a process where droplets of liquids, particles of solids or gasses are enclosed inside a solid shell and are generally in the micro-size range. The core material is then mechanically separated from the surrounding environment through a membrane (Jyothi et al., Journal of Microencapsulation, 2010, 27, 187-197). Microencapsulation technology is attracting attention from various fields of science and has a wide range of commercial applications for different industries. Overall, capsules are capable of one or more of (i) providing stability of a formulation or material via the mechanical separation of incompatible components, (ii) protecting the core material from the surrounding environment, (iii) masking or hiding an undesirable attribute of an active ingredient and (iv) controlling or triggering the release of the active ingredient to a specific time or location. All of these attributes can lead to an increase of the shelf-life of several products and a stabilization of the active ingredient in liquid formulations.

Encapsulation can be found in areas such as pharmaceuticals, personal care, textiles, food, coatings and agriculture. In addition, the main challenge faced by microencapsulation technologies in real-world commercial applications is that a complete retention of the encapsulated active within the capsule is required throughout the whole supply chain, until a controlled or triggered release of the core material is applied (Thompson et al., Journal of Colloid and Interface Science, 2015, 447, 217-228). There are significantly limited microencapsulation technologies that are safe for both the environment and human health with a long-term retention and active protection capability that can fulfill the needs of the industry nowadays, especially when it comes to encapsulation of small molecules.

Over the past several years, manufacturers have used core-shell encapsulation techniques to preserve actives, such as benefit agents, in harsh environments and to release them at the desired time, which may be during or after use of the finished goods, such as consumer goods or other industrial or agricultural goods. Among the several mechanisms that can be used for release of benefit agent, the one commonly relied upon is mechanical rupture of the capsule shell. Selection of mechanical rupture as the release mechanism constitutes another challenge to the manufacturer, as rupture must occur at specific desired times, even if the capsules are subject to mechanical stress prior to the desired release time.

Industrial interest for encapsulation technology has led to the development of several polymeric capsules chemistries which attempt to meet the requirements of biodegradability, low shell permeability, high deposition, targeted mechanical properties and rupture profile. Increased environmental concerns have put the polymeric capsules under scrutiny, therefore manufacturers have started investigating sustainable solutions for the encapsulation of benefit agents.

Biodegradable materials exist and are able to form delivery particles via coacervation, spray-drying or phase inversion precipitation. However, the delivery particles formed using these materials and techniques are highly porous and not suitable for aqueous compositions containing surfactant, since the benefit agent is prematurely released to the composition.

Non-leaky and performing delivery particles in aqueous surfactant-based compositions exist, however due to its chemical nature and cross-linking, they are not biodegradable.

Delivery particles are needed that are biodegradable, yet have high structural integrity so as to reduce leakage and resist damage from harsh environments.

### DETAILED DESCRIPTION OF THE INVENTION

In accordance with embodiments, delivery particles with improved biodegradability comprising a core substantially enclosed in a polymer wall, the core comprising a benefit agent and a partitioning modifier, and the polymer wall obtained by the reaction of polymerizable monomers, such as (meth)acrylate monomers, with (meth)acrylic-functionalized natural or synthetic biodegradable polymers using diverse initiators to initiate the polymerization of the wall. Examples of natural and synthetic biodegradable polymers include chitosan and polyvinyl alcohol respectively.

The present invention includes novel delivery particles produced from cross-linking biodegradable polymers with smaller monomers in order to enhance the bioavailability of the wall and the biodegradability of the overall delivery particle. Without being bound by theory, it is believed that the biodegradable polymers form a network that enhances the accessibility of the enzymes during degradation process, while the small monomers close the delivery particle structure making it compacted enough to protect the benefit agent in an aqueous surfactant-based composition.

### DEFINITIONS

As used herein “consumer product” means baby care, beauty care, fabric & home care, family care, feminine care, health care, snack and/or beverage products or devices intended to be used or consumed in the form in which it is sold, and not intended for subsequent commercial manufacture or modification. Such products include but are not limited to fine fragrances (e.g. perfumes, colognes eau de toilettes, after-shave lotions, pre-shave, face waters, tonics, and other fragrance-containing compositions for application directly to the skin), diapers, bibs, wipes; products for and/or methods relating to treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use; and shaving products, products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, and other cleaning for consumer or institutional use; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening; over-the-counter health care including cough and cold remedies, pain relievers, Rx pharmaceuticals, pet health and nutrition, and water purification.

As used herein, the term “cleaning composition” includes, unless otherwise indicated, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various pouches, tablet, granular, liquid and rinse-aid types for household and institutional use; liquid cleaning and disinfecting agents, including antibacterial hand-wash types, cleaning bars, mouthwashes, denture cleaners, dentifrice, car or carpet shampoos, bathroom cleaners; hair shampoos and hair-rinses; shower gels and foam baths and metal cleaners; as well as cleaning auxiliaries such as bleach additives and “stain-stick” or pre-treat types, substrate-laden products such as dryer added sheets, dry and wetted wipes and pads, nonwoven substrates, and sponges; as well as sprays and mists.

As used herein, the term “fabric care composition” includes, unless otherwise indicated, fabric softening compositions, fabric enhancing compositions, fabric freshening compositions and combinations thereof. The form of such compositions includes liquids, gels, beads, powders, flakes, and granules.

As used herein, the phrase “benefit agent containing delivery particle” encompasses microcapsules including an active material. Nonlimiting examples include perfume microcapsules, microcapsules with lubricants, and microcapsules with other actives as described herein.

As used herein, the terms “delivery particle”, “benefit agent containing delivery particle”, “encapsulated benefit agent”, “capsule” and “microcapsule” are synonymous.

As used herein, reference to the term “(meth)acrylate” or “(meth)acrylic” is to be understood as referring to both the acrylate and the methacrylate versions of the specified monomer, oligomer and/or prepolymer, (for example “allyl (meth)acrylate” indicates that both allyl methacrylate and allyl acrylate are possible, similarly reference to alkyl esters of (meth)acrylic acid indicates that both alkyl esters of acrylic acid and alkyl esters of methacrylic acid are possible, similarly poly(meth)acrylate indicates that both polyacrylate and polymethacrylate are possible).

For purposes of this application, the partitioning modifier is not considered a perfume raw material and thus it is not considered when calculating perfume compositions/formulations. Thus, the amount of partitioning modifier present is not used to make such calculations.

As used herein the term “water soluble material” means a material that has a solubility of at least 0.5% wt in water at 60° C.

As used herein the term “oil soluble” means a material that has a solubility of at least 0.1% wt in the core of interest at 50° C.

As used herein the term “oil dispersible” means a material that can be dispersed at least 0.1% wt in the core of interest at 50° C. without visible agglomerates.

As used herein, the articles including “a” and “an” when used in a claim, are understood to mean one or more of what is claimed or described.

As used herein, the terms “include”, “includes” and “including” are meant to be non-limiting.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the terms “site” or “site of attachment” or “point of attachment” all mean an atom (e.g. A) having an open valence within a chemical group or defined structural entity that is designated with a symbol (*-A) to indicate that the so-designated atom A connects to another atom in a separate chemical group via a covalent chemical bond.

As used herein, the symbol “ [symbol] ” when drawn perpendicular across a bond also indicates a point of attachment to the carbon-carbon polymer chain resulting from radical polymerization of a polymerizable functional group (e.g. the C—C double bond of meth(acrylate)).

As used herein “biodegradable” refers to a material that has above 30% CO₂ release according to the OECD301B test method.

### DELIVERY PARTICLE

The present disclosure relates to a composition that comprises a delivery particle as described in more detail below.

The composition of the present invention comprises a delivery particle of the core/shell type comprising a core and a polymer wall encapsulating said core, and comprises a population of delivery particles.

The polymer wall comprises at least one water soluble material of formula I

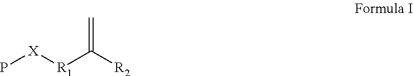

wherein
P is a polymer with a molecular weight of from about 30 kDa to about 500 kDa, preferably from about 50 kDa to about 300 kDa, even more preferably from about 80 kDa to about 200 kDa, selected from the group consisting of poly(vinyl alcohol), chitosan, chitin, pectin, carrageenan, xanthan gum, tara gum, gelatine, konjac gum, alginate, hyaluronic acid, amylose, lignin, diutan gum, and mixtures thereof;
X is a heteroatom covalently linked to the polymer; X is preferably O or N;
R₁ is independently selected from the group consisting of

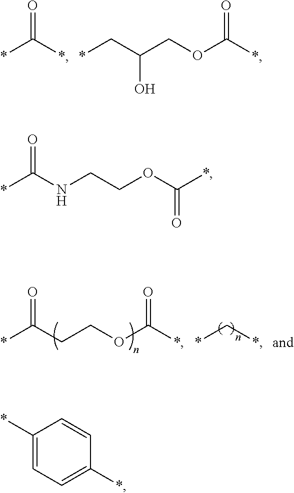

wherein m and n are integers independently selected from 1 to 100, preferably from 1 to 50, even more preferably from 1 to 10;
R₂ is independently selected from the group consisting of *H and *CH₃.

In embodiments, P is preferably selected from the group consisting of a polyvinyl alcohol polymer, a chitosan polymer, a chitin polymer, or mixtures thereof.

In embodiments, R₁ is preferably

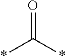 [SMILES: *C(*)=O]

and R₂ a hydrogen (*—H) or methyl group (*—CH₃).

In embodiments, the water soluble material is from about 20% wt to about 95% wt, at least 30% wt, at least 40% wt, at least 50% wt, preferably at least 75% wt, more preferably at least 85% wt, even more preferably 95% wt of the total polymer wall.

In embodiments, the water soluble material has a percentage of heteroatoms functionalized with a radical polymerizable group as depicted in FIG. 1 from 0.05 to 20% wt, preferably from 0.5 to 10% wt, even more preferably from 0.6 to 5% wt.

In embodiments, the water soluble material comprises at least one (meth)acrylate polymerizable group as depicted in FIG. 1 when R₂ is *—CH₃.

In embodiments, the water soluble material has a biodegradability in 60 days following OECD 301B test above 30% CO₂, preferably above 40% CO₂, more preferably above 50% CO₂, even more preferably above 60% CO₂ (maximum 100%).

In embodiments, the water soluble material is obtained by the (meth)acrylate functionalization of poly(vinyl alcohol), chitosan, chitin, pectin, carrageenan, gelatine, xanthan gum, tara gum, konjac gum, alginate, hyaluronic acid, amylose, lignin, diutan gum, and mixtures thereof, preferably by the methacrylate functionalization of poly(vinyl alcohol), chitosan, chitin, and mixtures thereof. In embodiments, the water soluble or water dispersible material is functionalized with (meth)acrylic anhydride, glycidyl (meth)acrylate, 2-isocyanatoethyl (meth)acrylate, 2,5-dioxopyrrolidin-1-yl (meth)acrylate, (meth)acrylic acid, and mixtures thereof, preferably with (meth)acrylic anhydride.

Non-limiting examples of water soluble or water dispersible material are

1. Methacrylate functionalized poly(vinyl alcohol)

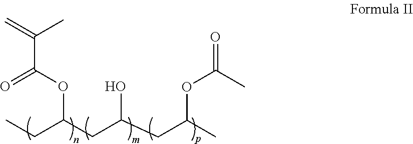

wherein n, m and p, are integers from 1 to 100000, with n, m, and p at least 340. The n:m:p molar ratio is from about 0.1:98.9:1 to about 10:60:30, preferably from about 0.2:91.8:8 to about 5:70:25, even more preferably from about 1:87:12 to about 5:80:15.

In embodiments the polymer P is derived from poly(vinyl alcohol) with a weight average molecular weight from about 30 kDa to about 500 kDa, preferably from about 50 kDa to about 300 kDa, even more preferably from about 80 kDa to about 200 kDa. The poly(vinyl alcohol) preferably has a hydrolysis degree from about 55% to about 99%, preferably from about 75% to about 95%, more preferably from about 85% to about 90%, and most preferably from about 87% to about 89%.

2. Methacrylate functionalized chitosan

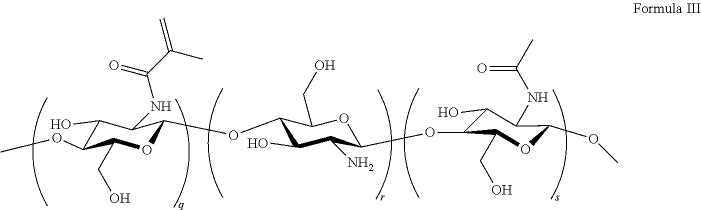

wherein q, r and s are integers from 1 to 2400, with q+r+s at least 146. The q:r:s molar ratio is from about 0.1:98.9:1 to about 10:10:80, preferably from about 0.2:90:9.8 to about 5:40:55, even more preferably from about 1:87:12 to about 5:80:15.

In embodiments, the polymer P is derived from nitrogen-containing polysaccharides, such as chitosan or chitin, with a degree of deacetylation (“DDA”) of at least 50%, preferably a DDA at least 65%, and more preferably a DDA at least 75%. In embodiments, the chitosan has a weight average molecular weight from about 30 kDa to about 500 kDa, preferably from about 50 kDa to about 300 kDa, even more preferably from about 80 kDa to about 200 kDa.

In embodiments, the polymer wall further comprises at least one multi-functional monomer and/or oligomer comprising more than one radical polymerizable functional group. In embodiments, the multi-functional monomer and/or oligomer comprises a radical polymerizable group selected from the group consisting of acrylate, methacrylate, styrene, allyl, vinyl, and mixtures thereof.

In embodiments, one or more oil-soluble or oil-dispersible multifunctional monomers or oligomers comprise at least two radical polymerizable functional groups, preferably at least three radical polymerizable functional groups, preferably at least four radical polymerizable functional groups, more preferably at least five radical polymerizable functional groups, even more preferably at least six radical polymerizable functional groups. In embodiments, one or more oil-soluble or oil-dispersible multifunctional (meth)acrylate monomers or oligomers comprise more than six radical polymerizable functional groups. It is believed that monomers comprising a greater number of radical polymerizable functional groups result in delivery particles with more compact walls that have preferred properties, such as less leakage, compared to walls formed from monomers that have fewer radical polymerizable functional groups.

In embodiments, at least two, or at least three, or at least four, or at least five, or at least six of the radical polymerizable functional groups are an acrylate or methacrylate group. Preferably, the radical polymerizable functional groups are each independently selected from the group consisting of acrylate and methacrylate. In embodiments, the radical polymerizable functional groups of the multi-functional monomer and/or oligomer are all the same. It is believed that these radical polymerizable functional groups result in delivery particles having preferred properties, such as less leakage at high core:wall ratios, compared to other functional groups. In embodiments delivery particles may have leakage values of below about 50% or below about 30%, as determined by the Leakage Test described in the TEST METHODS Section.

The oil-soluble or oil-dispersible multifunctional (meth)acrylate monomers or oligomers may comprise a multifunctional aromatic urethane acrylate. Preferably, the oil-soluble or oil-dispersible multifunctional (meth)acrylate monomers or oligomers comprises a hexafunctional aromatic urethane acrylate.

Additionally, or alternatively, the oil-soluble or oil-dispersible multifunctional (meth)acrylate monomers or oligomers may comprise a multifunctional aliphatic urethane acrylate.

The polymer wall may be derived from at least two different multifunctional (meth)acrylate monomers, for example first and second multifunctional (meth)acrylate monomers. The first multifunctional (meth)acrylate monomer may comprise a different number of radical polymerizable functional groups compared to the second multifunctional (meth)acrylate monomer.

For example, the first multifunctional (meth)acrylate monomer may comprise six radical polymerizable functional groups (e.g., hexafunctional), and the second multifunctional (meth)acrylate monomer may comprise less than six radical polymerizable functional groups, such as a number selected from two (e.g., difunctional), three (e.g., trifunctional), four (e.g., tetrafunctional), or five (e.g., pentafunctional), preferably five. In embodiments, the first and second multifunctional (meth)acrylate monomers may comprise the same number of radical polymerizable functional groups, such as six (e.g., both monomers are hexafunctional), although the respective monomers are characterized by different structures or chemistries. In embodiments, the first and second multifunctional (meth)acrylate monomers may comprise different number of radical polymerizable functional groups, such as six and two.

In addition to the oil-soluble or oil-dispersible multi-functional (meth)acrylate monomer or oligomer, the polymer wall may be further derived from a water-soluble or water-dispersible multifunctional (meth)acrylate monomer or oligomer, which may include a hydrophilic functional group. The water-soluble or water-dispersible multifunctional (meth)acrylate monomer or oligomer may be preferably selected from the group consisting of polyethylene glycol di(meth)acrylates, ethoxylated multi-functional (meth)acrylates, and mixtures thereof, for example trimethylolpropane tri(meth)acrylate, ethylene glycol di(meth)acrylate, di-, tri- and tetraethyleneglycol di(meth)acrylate, dipropylene glycol di(meth)acrylate, polyethylene glycol di(meth)acrylate, di(pentamethylene glycol) di(meth)acrylate, ethylene di(meth)acrylate, neopentyl glycol di(meth)acrylate, trimethylol propane tri(meth)acrylate, diglycerol di(meth)acrylate, neopentyl di(meth)acrylate, trimethylolpropane tri(meth)acrylate, polyethylene glycol di(meth)acrylate and dipropylene glycol di(meth)acrylate, and mixtures thereof.

In embodiments, the polymer wall may further comprise a monomer selected from an amine (meth)acrylate, an acidic (meth)acrylate, or a combination thereof.

Suitable amine (meth)acrylates for use in the particles of the present disclosure may include aminoalkyl acrylate or aminoalkyl methacrylate including, for example, but not by way of limitation, ethylaminoethyl acrylate, ethylaminoethyl methacrylate, aminoethyl acrylate, aminoethyl methacrylate, tertiarybutyl aminoethyl acrylate, tertiarybutyl aminoethyl methacrylate, t diethylamino acrylate, diethylamino methacrylate, diethylaminoethyl acrylate diethylaminoethyl methacrylate, dimethylaminoethyl acrylate and dimethylaminoethyl methacrylate. Preferably, the amine (meth)acrylate is aminoethyl acrylate or aminoethyl methacrylate, or tertiarybutyl aminoethyl methacrylate.

Suitable carboxy (meth)acrylates for use in particles of the present disclosure may include 2-carboxyethyl acrylate, 2-carboxyethyl methacrylate, 2-carboxypropyl acrylate, 2-carboxypropyl methacrylate, carboxyoctyl acrylate, carboxyoctyl methacrylate. Carboxy substituted aryl acrylates or methacrylates may include 2-acryloyloxybenzoic acid, 3-acryloyloxybenzoic acid, 4-acryloyloxybenzoic acid, 2-methacryloyloxybenzoic acid, 3 -methacryloyloxybenzoic acid, and 4-methacryloyloxybenzoic acid. (Meth)acryloyloxyphenylalkylcarboxy acids by way of illustration and not limitation can include 4-acryloyloxyphenylacetic acid or 4-methacryloyloxyphenylacetic acid.

In embodiments, the polymer wall may be further derived, at least in part, from at least one free radical initiator, preferably at least two free radical initiators, even more preferably at least three radical initiators. In embodiments, at least one free radical initiator may preferably comprise a water-soluble or water-dispersible free radical initiator. In embodiments, at least one free radical initiator may preferably comprise an oil-soluble or oil-dispersible free radical initiator. In a preferred embodiment, the polymer wall may be derived, at least in part, from the combination of at least one water-soluble or water-dispersible free radical initiator and at least one oil-soluble or oil-dispersible free radical initiator.

Without wishing to be bound by theory, it is believed that selecting the appropriate amount of initiator relative to total wall material (and/or wall monomers/oligomers) can result in improved capsules. For example, it is believed that levels of initiators that are too low may lead to poor polymer wall formation; levels that are too high may lead to encapsulate walls that have relatively low levels of structural monomers. In either situation, the resulting capsules may be relatively leaky and/or weak.

Thus, the amount of initiator present may be from about 0.1% to about 30%, preferably from about 0.5% to about 25%, more preferably from about 0.8% to about 15%, even more preferably from about 1% to about 10%, even more preferably from about 1% to about 8%, by weight of the polymer wall. It is believed that relatively higher amounts of initiator within the disclosed ranges may lead to improved, less-leaky capsules. The optimal amount of initiator may vary according to the nature of the core material. The polymer wall may be derived from a first initiator and a second initiator, wherein the first and second initiators are present in a weight ratio of from about 5:1 to about 1:5, or preferably from about 3:1 to about 1:3, or more preferably from about 2:1 to about 1:2, or even more preferably from about 1.5:1 to about 1:1.5.

Suitable free radical initiators may include azo initiators. More particularly, and without limitation, the free radical initiator can be selected from the group consisting of 2,2′-azobis(isobutylnitrile), 2,2′-azobis(2,4-dimethylpentanenitrile), 2,2′-azobis (2,4-dimethylvaleronitrile), 2,2′-azobis(2-methylpropanenitrile), 2,2′-azobis(2-methylbutyronitrile), 1,1′-azobis (cyclohexanecarbonitrile), 1,1′-azobis(cyanocyclohexane), and mixtures thereof.

The delivery particles of the present disclosure include a core. The core may comprise a benefit agent. Suitable benefit agents located in the core may include benefit agents that provide benefits to a surface, such as a fabric or hair, or to other surfaces or sites.

The core may comprise from about 40% to about 95%, preferably from about 50% to about 80%, more preferably from about 50% to about 70%, by weight of the core, of the benefit agent. The benefit agent can be an active material protected by the delivery particle, and intended delivered to a surface.

The benefit agent may be selected from the group consisting of fragrance, silicone oils, waxes, hydrocarbons, higher fatty acids, essential oils, lubricants, lipids, skin coolants, vitamins, sunscreens, antioxidants, glycerine, catalysts, bleach particles, silicon dioxide particles, malodor reducing agents, odor-controlling materials, chelating agents, antistatic agents, softening agents, insect and moth repelling agents, agricultural actives, plant nutrients, herbicides, fungicides, colorants, antioxidants, chelants, bodying agents, drape and form control agents, smoothness agents, wrinkle control agents, sanitization agents, disinfecting agents, germ control agents, mold control agents, mildew control agents, antiviral agents, drying agents, stain resistance agents, soil release agents, fabric refreshing agents and freshness extending agents, chlorine bleach odor control agents, dye fixatives, dye transfer inhibitors, color maintenance agents, optical brighteners, color restoration/rejuvenation agents, anti-fading agents, whiteness enhancers, anti-abrasion agents, wear resistance agents, fabric integrity agents, anti-wear agents, anti-pilling agents, defoamers, anti-foaming agents, UV protection agents, sun fade inhibitors, anti-allergenic agents, enzymes, water proofing agents, fabric comfort agents, shrinkage resistance agents, stretch resistance agents, stretch recovery agents, skin care agents, glycerin, synthetic or natural actives, antibacterial actives, antiperspirant actives, cationic polymers, dyes, and mixtures thereof. Preferably the benefit agent comprises fragrance, essential oils and mixtures thereof.

The encapsulated benefit agent may preferably be a fragrance, which may include one or more perfume raw materials. The term “perfume raw material” (or “PRM”) as used herein refers to compounds having a molecular weight of at least about 100 g/mol and which are useful in imparting an odor, fragrance, essence or scent, either alone or with other perfume raw materials. Typical PRMs comprise inter alia alcohols, ketones, aldehydes, esters, ethers, nitriles and alkenes, such as terpene. A listing of common PRMs can be found in various reference sources, for example, “Perfume and Flavor Chemicals”, Vols. I and II; Steffen Arctander Allured Pub. Co. (1994) and “Perfumes: Art, Science and Technology”, Miller, P. M. and Lamparsky, D., Blackie Academic and Professional (1994).

The PRMs may be characterized by their boiling points (B.P.) measured at the normal pressure (760 mm Hg), and their octanol/water partitioning coefficient (P), which may be described in terms of logP, determined according to the test method below. Based on these characteristics, the PRMs may be categorized as Quadrant I, Quadrant II, Quadrant III, or Quadrant IV perfumes, as described in more detail below.

The fragrance may comprise perfume raw materials that have a logP of from about 2.5 to about 4. It is understood that other perfume raw materials may also be present in the fragrance.

The perfume raw materials may comprise a perfume raw material selected from the group consisting of perfume raw materials having a boiling point (B.P.) lower than about 250° C. and a logP lower than about 3, perfume raw materials having a B.P. of greater than about 250° C. and a logP of greater than about 3, perfume raw materials having a B.P. of greater than about 250° C. and a logP lower than about 3, perfume raw materials having a B.P. lower than about 250° C. and a log greater than about 3, and mixtures thereof. Perfume raw materials having a boiling point B.P. lower than about 250° C. and a log lower than about 3 are known as Quadrant I perfume raw materials. Quadrant I perfume raw materials are preferably limited to less than 30% of the perfume composition. Perfume raw materials having a B.P. of greater than about 250° C. and a logP of greater than about 3 are known as Quadrant IV perfume raw materials, perfume raw materials having a B.P. of greater than about 250° C. and a logP lower than about 3 are known as Quadrant II perfume raw materials, perfume raw materials having a B.P. lower than about 250° C. and a logP greater than about 3 are known as a Quadrant III perfume raw materials. Suitable Quadrant I, II, III and IV perfume raw materials are disclosed in U.S. Pat. No. 6,869,923 B1.

The core of the delivery particles of the present disclosure may further comprise a partitioning modifier. The properties of the partitioning modifier in the core can play a role in determining how much, how quickly, and/or how permeable the polyacrylate shell material will be when established at the oil/water interface. For example, if the oil phase comprises highly polar materials, these materials may reduce the diffusion of the acrylate oligomers and polymers to the oil/water interface and result in a very thin, highly permeable shell. Incorporation of a partitioning modifier can adjust the polarity of the core, thereby changing the partition coefficient of the polar materials in the partitioning modifier versus the acrylate oligomers, and can result in the establishment of a well-defined, highly impermeable shell. The partitioning modifier may be combined with the core's benefit agent prior to incorporation of the wall-forming monomers.

The partitioning modifier may be present in the core at a level of from about 5% to about 60%, preferably from about 20% to about 50%, more preferably from about 30% to about 50%, by weight of the core.

The partitioning modifier may comprise a material selected from the group consisting of vegetable oil, modified vegetable oil, mono-, di-, and tri-esters of C₄-C₂₄ fatty acids, isopropyl myristate, dodecanophenone, lauryl laurate, methyl behenate, methyl laurate, methyl palmitate, methyl stearate, and mixtures thereof. The partitioning modifier may preferably comprise or even consist of isopropyl myristate. The modified vegetable oil may be esterified and/or brominated. The modified vegetable oil may preferably comprise castor oil and/or soybean oil. US Patent Application Publication 20110268802, incorporated herein by reference, describes other partitioning modifiers that may be useful in the presently described delivery particles.

In embodiments, the polymer wall is a cross-linked polymer comprising sub-units covalently linked by C—C bonds at designated points of attachment.

In embodiments, at least one sub-unit of the polymer wall has the formula IV

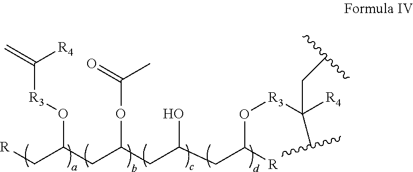

wherein
R₃ is a linking group independently selected from the group consisting of

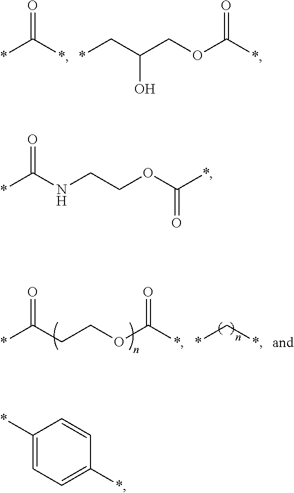

R₄ independently selected from the group consisting of hydrogen (*—H) and methyl (*—CH₃);
R is an end group independently selected from the group consisting of vinyl (*—CH═CH₂), hydrogen (*—H) and methyl (*—CH₃);
a is an integer from 0 to 5000, b, c and d are integers from 1 to 100000, with a+b+c+d at least 340; the a:b:c:d molar ratio is from about 0.0001:98.9:1:0.1 to about 1:59:30:10, preferably from about 0.001:91.8:8:0.2 to about 0.5:70:24.5:5, even more preferably from about 0.001:87:12:1 to about 0.1:79.9:15:5.

In embodiments, at least one sub-unit of the polymer wall has the formula V

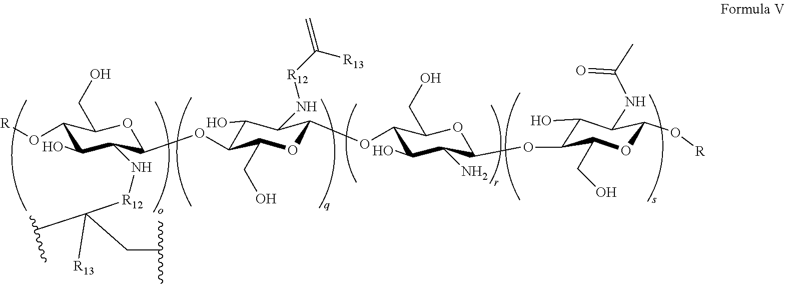

wherein
R₁₂ is a linking group independently selected from the group consisting of

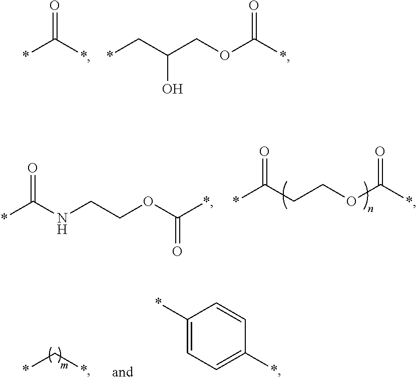

R₁₃ independently selected from the group consisting of hydrogen (*—H) and methyl (*—CH₃);
R is an end group independently selected from the group consisting of hydrogen (*—H) and methyl (*—CH₃);
q is an integer from 0 to 500, o, r and s are integers from 0 to 100000, with o+q+r+s at least 146; the o:q:r:s molar ratio is from about 0.1:0.0001:98.9:1 to about 10:1:9:80, preferably from about 0.2:0.001:90:9.8 to about 5:0.5:40:54.5, even more preferably from about 1:0.001:87:12 to about 5:0.05:80:14.95.

In embodiments, at least one sub-unit of the polymer wall has the formula VI

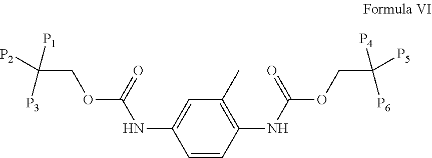

wherein
P₁, P₂, P₃, P₄, P₅ and P₆ can be independently selected from the group consisting of

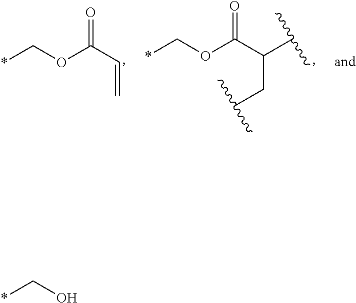

with the proviso that at least three of P₁, P₂, P₃, P₄, P₅ and P₆ are

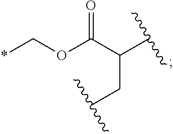 [SMILES: *COC(=O)C(*)C*]

and wherein the subunit of formula VI is at least 5% by weight of the total polymer wall.

In embodiments, at least one sub-unit of the polymer wall has the formula VII and/or VIII:

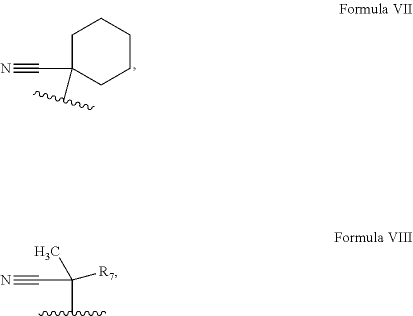

wherein R₇ is independently selected from the group consisting of

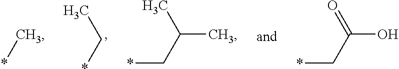

In embodiments, at least one sub-unit of the polymer wall has the formula IX and/or X:

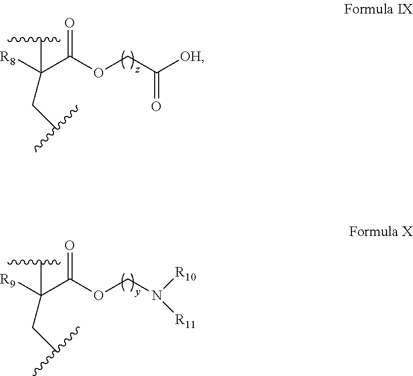

wherein
R₈ and R₉ are independently selected from the group consisting of a hydrogen (*—H) or a methyl group (*—CH₃);
z and y are integers independently selected from 1 to 10, preferably from 2 to 5,
R₁₀ and R₁₁ are independently selected from the group consisting of

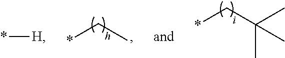

h and i are integers independently selected from 0 to 10, preferably from 1 to 5.

In embodiments, the polymer wall is formed combining sub-units of formulas IV, VI, VII, VIII, IX and X. In another aspect, the polymer wall is formed combining sub-units of formulas IV, VI, VII and/or VIII.

In embodiments, the polymer wall is formed combining sub-units of formulas V, VI, VII, VIII, IX and X. In another aspect, the polymer wall is formed combining sub-units of formulas V, VI, VII and/or VIII.

In embodiments, the weight ratio of Formula IX sub-units to Formula X sub-units is from about 10:1 to about 0.1:1, more preferably from about 5:1 to about 1:1, even more preferably from about 4:1 to about 1.5:1.

In embodiments, the polymer wall is formed combining sub-units of formulas IV, V, VI, VII, VIII, IX and X. In another aspect, the polymer wall is formed combining sub-units of formulas IV, V, VI, VII and/or VII.

In embodiments, the weight ratio of Formula IV sub-units to Formula VI sub-units is from about 20:70 to about 95:5, more preferably from about 30:60 to about 93:7, even more preferably from about 50:50 to about 90:10.

In embodiments, the weight ratio of Formula V sub-units to Formula VI sub-units is from about 20:70 to about 95:5, more preferably from about 30:60 to about 93:7, even more preferably from about 50:50 to about 90:10.

Delivery particles may be made according to known methods. Methods may be further adjusted to achieve desired characteristics described herein, such as volume-weighted particle size, relative amounts of benefit agent and/or partitioning modifier, etc.

For example, the present disclosure relates to a process of making a population of delivery particles comprising a core and a polymer wall encapsulating the core. The process may comprise the step of providing an oil phase. The oil phase may comprise a benefit agent and a partition modifier, as described above. The process may further comprise dissolving or dispersing into the oil phase one or more multifunctional monomers or oligomers having at least two, and preferably at least three, at least four, at least five, or even at least six radical polymerizable functional groups with the proviso that at least one of the radical polymerizable groups is acrylate or methacrylate.

The multifunctional monomers or oligomers are described in more detail above. Among other things, the multifunctional monomers or oligomers may comprise a multifunctional aromatic urethane acrylate, preferably a tri-, tetra-, penta-, or hexafunctional aromatic urethane acrylate, or mixtures thereof, preferably comprising a hexafunctional aromatic urethane acrylate. The monomer or oligomer may comprise one or more multifunctional aliphatic urethane acrylates, which may be dissolved or dispersed into the oil phase. The process may further comprise dissolving or dispersing one or more of an amine (meth)acrylate or an acidic (meth)acrylate into the oil phase.

The process further comprises a water phase comprising water soluble material that has Formula I (described above). In embodiments, the water soluble material of Formula I, is prepared in organic solvent and purified before used. In another aspect, the water soluble material of Formula I is prepared and used without purification using water as solvent.

The water phase may further comprise an emulsifier, a surfactant, or a combination thereof. The process may further comprise the step of dissolving or dispersing into the water phase one or more water-soluble or water-dispersible mono- or multi- functional (meth)acrylate monomers and/or oligomers.

The process may comprising a step of dissolving or dispersing in into the water phase, the oil phases, or both, one or more amine (meth)acrylates, acidic (meth)acrylates, polyethylene glycol di(meth)acrylates, ethoxylated mono- or multi-functional (meth)acrylates, and/or other (meth)acrylate monomers and/or oligomers.

In general, the oil soluble multifunctional monomer is soluble or dispersible in the oil phase, typically soluble at least to the extent of 0.1 grams in 100 ml of the oil, or dispersible or emulsifiable therein at 50° C. The water soluble multifunctional monomers are typically soluble or dispersible in water, typically soluble at least to the extent of 1 gram in 100 ml of water, or dispersible therein at 22° C.

Typically, the oil phase is combined with an excess of the water phase. If more than one oil phase is employed, these generally are first combined, and then combined with the water phase. If desired, the water phase can also comprise one or more water phases that are sequentially combined.

The oil phase may be emulsified into the water phase under high shear agitation to form an oil-in-water emulsion, which may comprise droplets of the core materials dispersed in the water phase. Typically, the amount of shear agitation applied can be controlled to form droplets of a target size, which influences the final size of the finished encapsulates.

The dissolved or dispersed monomers may be reacted by heating or actinic irradiation of the emulsion. The reaction can form a polymer wall at an interface of the droplets and the water phase. The radical polymerizable groups of the multifunctional monomer or oligomer, upon heating, facilitate self-polymerization.

One or more free radical initiators may be provided to the oil phase, the water phase, or both, preferably both. For example, the process may comprise adding one or more free radical initiators to the water phase, for example to provide a further source of free radicals upon activation by heat. The process may comprise adding one or more free radical initiators to the oil phase. The one or more free radical initiators may be added to the water phase, the oil phase, or both in an amount of from greater than 0% to about 5%, by weight of the respective phase. Latent initiators are also contemplated where a first action, particularly a chemical reaction, is needed to transform the latent initiator into an active initiator which subsequently initiates polymerization upon exposure to polymerizing conditions. Where multiple initiators are present, it is contemplated, and preferred, that each initiator be initiated or suitably initiated by a different condition.

Alternatively, the reacting step may be carried out in the absence of an initiator, as it has surprisingly been found that encapsulates may form, even when a free radical initiator is not present.

In the described process, the heating step may comprise heating the emulsion from about 1 hour to about 20 hours, preferably from about 2 hours to about 15 hours, more preferably about 4 hours to about 10 hours, most preferably from about 5 to about 7 hours, thereby heating sufficiently to transfer from about 500 joules/kg to about 5000 joules/kg to said emulsion, from about 1000 joules/kg to about 4500 joules/kg to said emulsion, from about 2900 joules/kg to about 4000 joules/kg to said emulsion.

Prior to the heating step, the emulsion may be characterized by a volume-weighted median particle size of the emulsion droplets of from about 0.5 microns to about 100 microns, even from about 1 microns to about 60 microns, or even from 20 to 50 microns, preferably from about 30 microns to about 50 microns, with a view to forming a population of delivery particles with a volume-weighted target size, for example, of from about 30 to about 50 microns.

The benefit agent may be selected as described above and is preferably a fragrance that comprises one or more perfume raw materials. The benefit agent may be the primary, or even only component, of the oil phase into which the other materials are dissolved or dispersed.

The partitioning modifier may be selected from the group consisting of isopropyl myristate, vegetable oil, modified vegetable oil, mono-, di-, and tri-esters of C4-C24 fatty acids, dodecanophenone, lauryl laurate, methyl behenate, methyl laurate, methyl palmitate, methyl stearate, and mixtures thereof, preferably isopropyl myristate. The partitioning modifier may be provided in an amount so as to comprise from about 5% to about 60% by weight of the core of the delivery particle.

As a result of the method of making delivery particles provided herein, the delivery particles may be present in an aqueous slurry, for example, the particles may be present in the slurry at a level of from about 10% to about 60%, preferably from about 20% to about 50%, by weight of the slurry. Additional materials may be added to the slurry, such as preservatives, solvents, structurants, or other processing or stability aids. The slurry may comprise one or more perfumes (i.e., unencapsulated perfumes) that are different from the perfume or perfumes contained in the core of the benefit agent delivery particles.

As discussed previously, an emulsion is formed by emulsifying under high shear agitation the oil or combined oils into the water phase. Optionally the water phase can also include emulsifiers. The water phase emulsifier can be selected form one or more of polyalkylene glycol ether, condensation products of alkyl phenols, aliphatic alcohols, or fatty adds with alkylene oxide, ethoxylated alkyl phenols, ethoxylated arylphenols, ethoxylated polyaryl phenols, carboxylic esters solubilized with a polyol, polyvinyl alcohol, polyvinyl acetate, or copolymers of polyvinyl alcohol polyvinyl acetate, polyacrylamide, poly(N-isopropylacrylamide), poly(2-hydroxypropyl methacrylate), poly(2-ethyl-2-oxazoline), poly(2-isopropenyl-2-oxazoline-co-methyl methacrylate), poly(methyl vinyl ether), and polyvinyl alcohol-co-ethylene). Especially useful polyvinyl alcohols include polyvinyl alcohols of molecular 13,000 to 1,876,000 Daltons, preferably from 13,000 to about 230,000 Daltons, or even from 146,000 to 186,000 Daltons. The polyvinyl alcohol can be partially or fully hydrolyzed. Polyvinyl alcohol partially hydrolyzed in the range of 80 to 95% hydrolyzed is preferred, even more preferred 87% to 89%.

Optionally, deposition aids can be included, or applied as a coating in one or more layers over formed or forming delivery particles, to increase deposition or adhesion of the delivery particles to various surfaces such as various substrates including but not limited to paper, fabric skin, hair, towels, or other surfaces. Deposition aids can include poly(meth)acrylate, poly-(ethylene-maleic anhydride), polyamine, wax, polyvinylpyrrolidone, polyvinylpyrrolidone co-polymers, polyvinylpyrrolidone-ethyl acrylate, polyvinylpyrrolidone-vinyl acrylate, polyvinylpyrrolidone methylacrylate, polyvinylpyrrolidone-vinyl acetate, polyvinyl acetal, polyvinyl butyral, polysiloxane, poly(propylene maleic anhydride), maleic anhydride derivatives, co-polymers of maleic anhydride derivatives, polyvinyl alcohol, styrene-butadiene latex, gelatin, gum Arabic, carboxymethyl cellulose, carboxymethyl hydroxyethyl cellulose, hydroxyethyl cellulose, other modified celluloses, sodium alginate, chitosan, casein, pectin, modified starch, polyvinyl acetal, polyvinyl butyral, polyvinyl methyl ether/maleic anhydride, polyvinyl pyrrolidone and its co polymers, poly(vinyl pyrrolidone/methacrylamidopropyl trimethyl ammonium chloride), polyvinylpyrrolidone/vinyl acetate, polyvinyl pyrrolidone/dimethylaminoethyl methacrylate, polyvinyl amines, polyvinyl formamides, polyallyl amines and copolymers of polyvinyl amines, polyvinyl formamides, and polyallyl amines and mixtures thereof. In a further embodiment, the above-described delivery particles can comprise a deposition aid, and in a further aspect the deposition aid coats the outer surface of the wall of the delivery particle.

### Slurries

The benefit agent delivery particle composition can comprise a slurry in the form of benefit agent delivery particles dispersed in a first aqueous carrier. The level and species of the carrier are selected according to the compatibility with other components, and other desired characteristic of the product. Accordingly, the formulations of the composition can be in the form of pourable liquids (under ambient conditions). Such compositions will therefore typically comprise a first aqueous carrier, which is present at a level of at least 20 wt %, from about 20 wt % to about 95 wt %, or from about 60 wt % to about 85 wt %. The first aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

### DEPOSITION AIDS

The delivery particle compositions of the present invention may further comprise a deposition aid, such as a cationic polymer as a coating on the particle or as an additive to a slurry. Cationic polymers useful herein are those having an average molecular weight of at least about 5,000, alternatively from about 10,000 to about 10 million, and alternatively from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

### SUSPENDING AGENTS

In one embodiment, the delivery particle composition comprises a rheology modifier of the aqueous slurry. The rheology modifier increases the substantivity and stability of the composition. Any suitable rheology modifier can be used. In an embodiment, the composition may comprise from about 0.05% to about 10% of a rheology modifier, in a further embodiment, from about 0.1% to about 10% of a rheology modifier, in yet a further embodiment, from about 0.5% to about 2% of a rheology modifier, in a further embodiment, from about 0.7% to about 2% of a rheology modifier, and in a further embodiment from about 1% to about 1.5% of a rheology modifier. In an embodiment, the rheology modifier may be a polyacrylamide thickener. In an embodiment, the rheology modifier may be a polymeric rheology modifier.

In an embodiment, the composition of the present invention may comprise suspending agents including crystalline suspending agents which can be categorized as acyl derivatives, long chain amine oxides, and mixtures thereof. These suspending agents are described in U.S. Pat. No. 4,741,855. These suspending agents include ethylene glycol esters of fatty acids in one aspect having from about 16 to about 22 carbon atoms. In embodiments, useful suspending agents include ethylene glycol stearates, both mono and distearate, but in one aspect, the distearate containing less than about 7% of the mono stearate. Other suitable suspending agents include alkanol amides of fatty acids, having from about 16 to about 22 carbon atoms, or even about 16 to 18 carbon atoms, examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin® R available from Rheox, Inc. Long chain acyl derivatives, ethylene glycol esters of long chain carboxylic acids, long chain amine oxides, and alkanol amides of long chain carboxylic acids in addition to the materials listed above may be used as suspending agents. Other long chain acyl derivatives suitable for use as suspending agents include N,N-dihydrocarbyl amido benzoic acid and soluble salts thereof (e.g., Na, K), particularly N,N-di(hydrogenated) C16, C18 and tallow amido benzoic acid species of this family, which are commercially available from Stepan Company (Northfield, Ill., USA). Examples of suitable long chain amine oxides for use as suspending agents include alkyl dimethyl amine oxides, e.g., stearyl dimethyl amine oxide. Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

### CLEANING COMPOSITIONS

The delivery particle of the current invention can be used in cleaning compositions to provide one or more benefits, including freshness and/or softness. The cleaning compositions of the present invention can be in different forms. Non-limiting examples of said forms are: soap, detergent, moisturizing body wash, gels, cleansers, cleansing milks, cleaning compositions used in conjunction with a disposable cleaning cloth, sprays, liquids, pastes, Newtonian or non-Newtonian fluids, gels, and sols.

The cleaning composition preferably comprises delivery particles at least comprising one benefit agent at a level where upon directed use, promotes one or more benefits. In one embodiment of the present invention, said cleaning composition comprises between about 0.01 wt % to about 15 wt % of at least one benefit agent encapsulated in said delivery particle. In another embodiment, said cleaning composition comprises between about 0.05% to about 8% of at least one benefit agent encapsulated. In another embodiment, said cleaning composition comprises between about 0.1% to about 5% of at least one benefit agent encapsulated.

In addition to at least one delivery particle, the cleaning compositions of the present invention may also include additional ingredients.

Cleaning compositions can be multi-phase or single phase. A cleaning composition can comprise a cleaning phase and a benefit phase. The cleaning phase and the benefit phase can be blended. The cleaning phase and the benefit phase can also be patterned (e.g. striped and/or marbled). In embodiments, the cleaning phase may comprise the delivery particle. In embodiments, the benefit phase may comprise the delivery particle.

### Additional Ingredients

Additional ingredients can also be added to the compositions for treatment or to modify the aesthetics of the composition as is the case with perfumes, colorants, dyes or the like. Materials useful in products herein can be categorized or described by their cosmetic and/or therapeutic benefit or their postulated mode of action or function. However, it can be understood that actives and other materials useful herein can, in some instances, provide more than one cosmetic and/or therapeutic benefit or function or operate via more than one mode of action. Therefore, classifications herein can be made for convenience and cannot be intended to limit an ingredient to particularly stated application or applications listed. A precise nature of these additional materials, and levels of incorporation thereof, will depend on the physical form of the composition and the nature of the cleansing operation for which it is to be used. The additional materials can usually be formulated at about 6% or less, about 5% or less, about 4% or less, about 3% or less, about 2% or less, about 1% or less, about 0.5% or less, about 0.25% or less, about 0.1% or less, about 0.01% or less, or about 0.005% or less of the rinse-off personal care composition.

To further improve stability under stressful conditions such as high temperature and vibration, densities of separate phases can be adjusted such that they can be substantially equal. To achieve this, low density microspheres can be added to one or more phases of the rinse-off personal care composition. Examples of rinse-off personal care compositions that comprise low density microspheres are described in a patent application published on May 13, 2004 under U.S. Patent Publication No. 2004/0092415A1 entitled “Striped Liquid Personal Cleansing Compositions Containing A Cleansing Phase and A Separate Phase with Improved Stability,” filed on Oct. 31, 2003 by Focht, et al.

Other non-limiting ingredients that can be used in addition in the composition of the present invention can comprise an optional benefit component that can be selected from the group consisting of thickening agents; preservatives; antimicrobials; fragrances; chelators (e.g. such as those described in U.S. Pat. No. 5,487,884 issued to Bisset, et al.); sequestrants; vitamins (e.g. Retinol); vitamin derivatives (e.g. tocophenyl acetate, niacinamide, panthenol); sunscreens; desquamation actives (e.g. such as those described in U.S. Pat. Nos. 5,681,852 and 5,652,228 issued to Bisset); anti-wrinkle/anti-atrophy actives (e.g. N-acetyl derivatives, thiols, hydroxyl acids, phenol); anti-oxidants (e.g. ascorbic acid derivatives, tocopherol) skin soothing agents/skin healing agents (e.g. panthenoic acid derivatives, aloe vera, allantoin); skin lightening agents (e.g. kojic acid, arbutin, ascorbic acid derivatives) skin tanning agents (e.g. dihydroxyacteone); anti-acne medicaments; essential oils; sensates; pigments; colorants; pearlescent agents; interference pigments (e.g. such as those disclosed in U.S. Pat. No. 6,395,691 issued to Liang Sheng Tsaur, U.S. Pat. No. 6,645,511 issued to Aronson, et al., U.S. Pat. No. 6,759,376 issued to Zhang, et al, U.S. Pat. No. 6,780,826 issued to Zhang, et al.) particles (e.g. talc, kaolin, mica, smectite clay, cellulose powder, polysiloxane, silicas, carbonates, titanium dioxide, polyethylene beads) hydrophobically modified non-platelet particles (e.g. hydrophobically modified titanium dioxide and other materials described in a commonly owned, patent application published on Aug. 17, 2006 under Publication No. 2006/0182699A, entitled “Personal Care Compositions Containing Hydrophobically Modified Non-platelet particle filed on Feb. 15, 2005 by Taylor, et al.) and mixtures thereof. The multiphase personal care composition can comprise from about 0.1% to about 4%, by weight of the rinse-off personal care composition, of hydrophobically modified titanium dioxide. Other such suitable examples of such skin actives are described in U.S. patent application Ser. No. 13/157,665.

### OTHER ARTICLES OF MANUFACTURE

This invention describes compositions comprising delivery particles, microcapsules, capsule manufacturing processes and microcapsules produced by such processes, along with improved articles of manufacture based on such microcapsules.

The microcapsules of the invention can be incorporated dry, as an aqueous slurry, as a coating or as a gel into a variety of commercial products to yield novel and improved articles of manufacture, including incorporation into or onto foams, mattresses, bedding, cushions, added to cosmetics or to medical devices, incorporated into or onto packaging, dry wall, construction materials, heat sinks for electronics, cooling fluids, incorporated into insulation, used with lotions, incorporated into gels including gels for coating fabrics, automotive interiors, and other structures or articles, including clothing, footwear, personal protective equipment and any other article where use of the improved capsules of the invention is deemed desirable.

More particularly and alternative to fragrances, the capsules of the invention for example are also suitable for various agriculture applications such as degradable delivery particles for delivering herbicides, various agricultural formulation, pesticides, pheromones, insect control agents or other actives. The articles of manufacture can be selected from the group consisting of a soap, a surface cleaner, a laundry detergent, a fabric softener, a shampoo, a textile, a paper towel, an adhesive, a wipe, a diaper, a feminine hygiene product, a facial tissue, a pharmaceutical, a napkin, a deodorant, a foam, a pillow, a mattress, bedding, a cushion, a cosmetic, a medical device, an agricultural product, packaging, a cooling fluid, a wallboard, and insulation.

The microcapsules facilitate improving flowability of encapsulated materials enhancing ease of incorporation into or onto articles such as foams, gels, textiles, various cleaners, detergents or fabric softeners. The microcapsules can be used neat, or more often blended into coatings, gels or used as an aqueous slurry or blended into other articles to form new and improved articles of manufacture.

### AGRICULTURAL FORMULATIONS

The delivery particles protect and separate the core material such as agricultural active or fragrance or other core material or benefit agent, keeping it separated from the external environment. This facilitates design of distinct and improved articles of manufacture.

In particular the delivery particles of the invention find applicability in a variety of articles of manufacture which in particular can include various agricultural formulations, including a slurry encapsulating an agricultural active, a population of dry microcapsules encapsulating an agricultural active, delivery particles of an encapsulated insecticide, and or delivery particles encapsulating an agricultural formulation for delivering a herbicide such as a preemergent herbicide. Advantageously the delivery particles of the invention desirably promote eventual degradation of such encapsulates or portions of the articles of manufacture. All such variations are contemplated herein with the disclosed delivery particles and microcapsules of the invention.

With other types of articles, for example, with phase change benefit agents, the microcapsules help preserve the repeated activity of the phase change material and retain the phase change material to prevent leakage or infusion into nearby components when isolation of the microcapsules is desired, yet promote eventual degradation of such encapsulates or portions of the articles of manufacture.

In agricultural applications, the microcapsules of the invention assist with targeted delivery to a surface or plant, protecting the benefit agent such as an agricultural active, herbicide or nutrient until delivered to the site of application and/or released

### FABRIC CARE COMPOSITIONS

Fabric care compositions of the present invention may include additional adjunct ingredients that include: bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, suds suppressors, dyes, additional perfumes and perfume delivery systems, structure elasticizing agents, fabric softeners, carriers, hydrotropes, processing aids, structurants, anti-agglomeration agents, coatings, formaldehyde scavengers and/or pigments. Other variants of Applicants' compositions do not contain one or more of the following adjuncts materials: bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, suds suppressors, dyes, additional perfumes and perfume delivery systems, structure elasticizing agents, fabric softeners, carriers, hydrotropes, processing aids, structurants, anti-agglomeration agents, coatings, formaldehyde scavengers, malodor reduction materials and/or pigments. The precise nature of these additional components, and levels of incorporation thereof, will depend on the physical form of the composition and the nature of the operation for which it is to be used. However, when one or more adjuncts are present, such one or more adjuncts may be present as detailed below. The following is a non-limiting list of suitable additional adjuncts.

### Deposition Aid

The fabric care composition may comprise from about 0.01% to about 10%, from about 0.05 to about 5%, or from about 0.15 to about 3% of a deposition aid. The deposition aid may be a cationic or amphoteric polymer. The deposition aid may be a cationic polymer. Cationic polymers in general and their method of manufacture are known in the literature. The cationic polymer may have a cationic charge density of from about 0.005 to about 23 meq/g, from about 0.01 to about 12 meq/g, or from about 0.1 to about 7 meq/g, at the pH of the composition. For amine-containing polymers, wherein the charge density depends on the pH of the composition, charge density is measured at the intended use pH of the product. Such pH will generally range from about 2 to about 11, more generally from about 2.5 to about 9.5. Charge density is calculated by dividing the number of net charges per repeating unit by the molecular weight of the repeating unit. The positive charges may be located on the backbone of the polymers and/or the side chains of polymers.

The weight-average molecular weight of the polymer may be from about 500 Daltons to about 5,000,000 Daltons, or from about 1,000 Daltons to about 2,000,000 Daltons, or from about 2,500 Daltons to about 1,500,000 Daltons, as determined by size exclusion chromatography relative to polyethylene oxide standards with RI detection. The weight-average molecular weight of the cationic polymer may be from about 500 Daltons to about 37,500 Daltons,

### Surfactants

Surfactants utilized can be of the anionic, nonionic, zwitterionic, ampholytic or cationic type or can comprise compatible mixtures of these types. Anionic and nonionic surfactants are typically employed if the fabric care product is a laundry detergent. On the other hand, cationic surfactants are typically employed if the fabric care product is a fabric softener. In addition to the anionic surfactant, the fabric care compositions of the present invention may further contain a nonionic surfactant. The compositions of the present invention can contain up to about 30%, alternatively from about 0.01% to about 20%, more alternatively from about 0.1% to about 10%, by weight of the composition, of a nonionic surfactant. The nonionic surfactant may comprise an ethoxylated nonionic surfactant. Suitable for use herein are the ethoxylated alcohols and ethoxylated alkyl phenols of the formula R(OC₂H₄)n OH, wherein R is selected from the group consisting of aliphatic hydrocarbon radicals containing from about 8 to about 20 carbon atoms and alkyl phenyl radicals in which the alkyl groups contain from about 8 to about 12 carbon atoms, and the average value of n is from about 5 to about 15.

The fabric care compositions of the present invention may contain up to about 30%, alternatively from about 0.01% to about 20%, more alternatively from about 0.1% to about 20%, by weight of the composition, of a cationic surfactant. For the purposes of the present invention, cationic surfactants include those which can deliver fabric care benefits. Non-limiting examples of useful cationic surfactants include: fatty amines; quaternary ammonium surfactants; and imidazoline quat materials.

Non-limiting examples of fabric softening actives are N,N-bis(stearoyl-oxy-ethyl) N,N-dimethylammonium chloride; N,N-bis(tallowoyl-oxo-ethyl) N,N-dimethylammonium chloride, N,N-bis(stearoyl-oxy-ethyl)N-(2 hydroxyethyl)N-methyl ammonium methyl sulfate; 1,2 di (stearoyl-oxy) 3 trimethyl ammoniumpropane chloride; dialkylenedimethylammonium salts such as dicanoladimethylammonium chloride, di(hard)tallowdimethylammonium chloride dicanoladimethylammonium methyl sulfate; 1-methyl-1-stearoylamidoethyl-2-stearoylimidazolinium methyl sulfate; 1-tallowylamidoethyl-2-tallowylimidazoline; N,N″-dialkyldiethylenetriamine; the reaction product of N-(2-hydroxyethyl)-1,2-ethylenediamine or N-(2-hydroxyisopropyl)-1,2-ethylenediamine with glycolic acid, esterified with fatty acid, where the fatty acid is (hydrogenated) tallow fatty acid, palm fatty acid, hydrogenated palm fatty acid, oleic acid, rapeseed fatty acid, hydrogenated rapeseed fatty acid; polyglycerol esters (PGEs), oily sugar derivatives, and wax emulsions and a mixture of the above.

It will be understood that combinations of softener actives disclosed above are suitable fur use herein.

### Builders

The compositions, for example, if the delivery system particles are assembled into a detergent, may also contain from about 0.1% to 80% by weight of a builder. Compositions in liquid form generally contain from about 1% to 10% by weight of the builder component. Compositions in granular form generally contain from about 1% to 50% by weight of the builder component. Detergent builders are well known in the art and can contain, for example, phosphate salts as well as various organic and inorganic nonphosphorus builders. Water-soluble, nonphosphorus organic builders useful herein include the various alkali metal, ammonium and substituted ammonium polyacetates, carboxylates, polycarboxylates and polyhydroxy sulfonates. Examples of polyacetate and polycarboxylate builders are the sodium, potassium, lithium, ammonium and substituted ammonium salts of ethylene diamine tetraacetic acid, nitrilotriacetic acid, oxydisuccinic acid, mellitic acid, benzene polycarboxylic acids, and citric acid. Other polycarboxylate builders are the oxydisuccinates and the ether carboxylase builder compositions comprising a combination of tartrate monosuccinate and tartrate disuccinate. Builders for use in liquid detergents include citric acid. Suitable nonphosphorus, inorganic builders include the silicates, aluminosilicates, borates and carbonates, such as sodium and potassium carbonate, bicarbonate, sesquicarbonate, tetraborate decahydrate, and silicates having a weight ratio of SiO2 to alkali metal oxide of from about 0.5 to about 4.0, or from about 1.0 to about 2.4. Also useful are aluminosilicates including zeolites.

### Dispersants

The compositions may contain from about 0.1%, to about 10%, by weight of dispersants. Suitable water-soluble organic materials are the homo- or co-polymeric acids or their salts, in which the polycarboxylic acid may contain at least two carboxyl radicals separated from each other by not more than two carbon atoms. The dispersants may also be alkoxylated derivatives of polyamines, and/or quaternized derivatives.

### Enzymes

The compositions may contain one or more detergent enzymes which provide cleaning performance and/or fabric care benefits. Examples of suitable enzymes include hemicellulases, peroxidases, proteases, cellulases, xylanases, lipases, phospholipases, esterases, cutinases, pectinases, keratanases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination may be a cocktail of conventional applicable enzymes like protease, lipase, cutinase and/or cellulase in conjunction with amylase. Enzymes can be used at their art-taught levels, for example at levels recommended by suppliers such as Novozymes and Genencor. Typical levels in the compositions are from about 0.0001% to about 5%. When enzymes are present, they can be used at very low levels, e.g., from about 0.001% or lower; or they can be used in heavier-duty laundry detergent formulations at higher levels, e.g., about 0.1% and higher. In accordance with a preference of some consumers for “non-biological” detergents, the compositions may be either or both enzyme-containing and enzyme-free.

### Dye Transfer Inhibiting Agents

The compositions may also include from about 0.0001%, from about 0.01%, from about 0.05% by weight of the compositions to about 10%, about 2%, or even about 1% by weight of the compositions of one or more dye transfer inhibiting agents such as polyvinylpyrrolidone polymers, polyamine N-oxide polymers, copolymers of N-vinylpyrrolidone and N-vinylimidazole, polyvinyloxazolidones and polyvinylimidazoles or mixtures thereof.

### Chelant

The compositions may contain less than about 5%, or from about 0.01% to about 3% of a chelant such as citrates; nitrogen-containing, P-free aminocarboxylates such as EDDS, EDTA and DTPA; aminophosphonates such as diethylenetriamine pentamethylenephosphonic acid and, ethylenediamine tetramethylenephosphonic acid; nitrogen-free phosphonates e.g., HEDP; and nitrogen or oxygen containing, P-free carboxylate-free chelants such as compounds of the general class of certain macrocyclic N-ligands such as those known for use in bleach catalyst systems.

### Bleach System

Bleach systems suitable for use herein contain one or more bleaching agents. Non-limiting examples of suitable bleaching agents include catalytic metal complexes; activated peroxygen sources; bleach activators; bleach boosters; photobleaches; bleaching enzymes; free radical initiators; H₂O₂; hypohalite bleaches; peroxygen sources, including perborate and/or percarbonate and combinations thereof. Suitable bleach activators include perhydrolyzable esters and perhydrolyzable imides such as, tetraacetyl ethylene diamine, octanoylcaprolactam, benzoyloxybenzenesulphonate, nonanoyloxybenzene-sulphonate, benzoylvalerolactam, dodecanoyloxybenzenesulphonate. Other bleaching agents include metal complexes of transitional metals with ligands of defined stability constants.

### Stabilizer

The compositions may contain one or more stabilizers and thickeners. Any suitable level of stabilizer may be of use; exemplary levels include from about 0.01% to about 20%, from about 0.1% to about 10%, or from about 0.1% to about 3% by weight of the composition. Non-limiting examples of stabilizers suitable for use herein include crystalline, hydroxyl-containing stabilizing agents, trihydroxystearin, hydrogenated oil, or a variation thereof, and combinations thereof. In some aspects, the crystalline, hydroxyl-containing stabilizing agents may be water-insoluble wax-like substances, including fatty acid, fatty ester or fatty soap. In other aspects, the crystalline, hydroxyl-containing stabilizing agents may be derivatives of castor oil, such as hydrogenated castor oil derivatives, for example, castor wax. Other stabilizers include thickening stabilizers such as gums and other similar polysaccharides, for example gellan gum, carrageenan gum, and other known types of thickeners and rheological additives. Exemplary stabilizers in this class include gum-type polymers (e.g. xanthan gum), polyvinyl alcohol and derivatives thereof, cellulose and derivatives thereof including cellulose ethers and cellulose esters and tamarind gum (for example, comprising xyloglucan polymers), guar gum, locust bean gum (in some aspects comprising galactomannan polymers), and other industrial gums and polymers.

### Silicones

Suitable silicones comprise Si—O moieties and may be selected from (a) non-functionalized siloxane polymers, (b) functionalized siloxane polymers, and combinations thereof. The molecular weight of the organosilicone is usually indicated by the reference to the viscosity of the material. The organosilicones may comprise a viscosity of from about 10 to about 2,000,000 centistokes at 25° C. Suitable organosilicones may have a viscosity of from about 10 to about 00,000 centistokes at 25° C.

### TEST METHODS

It is understood the test methods disclosed in the TEST METHODS Section should be used to determine the respective values of the parameters described and claimed in the present application.

### Degree of Substitution Determination via ¹H-NMR

To determine the degree of substitution of the water soluble material, proton Nuclear Magnetic Resonance (NMR) spectrophotometry is used.

The water soluble material samples are prepared in DMSO-d6 (Cambridge Isotopes, Andover, MA) solvent with concentration 5 mg/ml. ¹H-NMR spectra is recorded on a 400 MHz Avance II—Deimos (from Bruker), with a 5 mm dual channel probe head (¹H and Broadband (BBO-type)) and a fully automated bacs sample changer for 60 samples. Analyses is run with Topspin 2.1 in ICONNMR environment. Spectrum processing is done with Topspin 4.0.5 software.

The area under the signal is proportional to the number of protons that signal corresponds to, for example in ¹H spectra of a water soluble material with a poly(vinyl alcohol) backbone the signal for the methacrylate protons fall at δ(ppm)˜5.6 and 6.

The degree of substitution is determined by comparing the integral intensities of corresponding groups in the NMR spectrum. It is important to realize that the compositional information represents a global average and provides no details regarding the compositional heterogeneity within the population of polymer chains.

Despite the spectra will be unique for each polymer, for example, in order to determine the degree of substitution of a water soluble material with a poly(vinyl alcohol) backbone the area for the hydrogens corresponding to the methacrylate groups (CH) and poly(vinyl alcohol) (CH₂) is integrated:

$$Degree of substitution ( \% ) = \frac{{A}_{H} \times 100}{A {CH}_{2}}$$

wherein,
A_{H} is the total area of the methacrylate groups (CH) (addition of the area at δ(ppm)˜5.6 and 6)
ACH₂ is the area corresponding to the poly(vinyl alcohol) (CH₂) at δ(ppm)˜1.2-1.8

### Water Soluble Material Determination in the Continuous Phase Based on Total Continuous Phase Water Weight

Free water soluble material level is determined by using Capillary Gel Permeation Chromatography-Quadrupole Time-Of-Flight Mass Spectrometry (CapGPC-QTOFMS).

The method is based on measuring the total ion intensity of specific fragment ion(s) from the water soluble material structure generated in electrospray quadrupole time-of-flight mass spectrometer operated at an elevated collision energy (CE). These common fragment ions are different and specific for each water soluble material of interest in a sample. The total ion intensity correlates with the water soluble material concentration in the delivery particles' population slurry sample solutions. It is considered that the amount of water soluble polymer that could not be recovered in the wash water is bounded to the polymer shell.

### Experimental Conditions

### Calibration Solution Preparation

5% water soluble material stock solution is first diluted to give 0.05% solution with de-ionized water (Millipore), then further a 2-fold serial dilution to cover the concentration range of interest, e.g., 0.00078%-0.05%.

### Sample Solution Preparation

Each delivery particles slurry is first diluted with water by 5 or 10 times, then delivery particles are separated from the aqueous phase via centrifugation and aqueous phase is filtered by passing them through 0.45 μm PVDF membrane filters (PALL Gelman Lab) and analyzed. This process is repeated 3 times to ensure all free water soluble material is extracted from the delivery particles slurry.

### Capillary Gel Permeation Chromatography-Mass Spectrometric Analysis (“CapGPC”)

Both calibration standard solutions and sample solutions are analyzed by injecting 5 μl onto a GPC system coupled to electrospray quadrupole time-of-flight mass spectrometer (Synapt QTOF, Waters, Beverly, Mass.) with four 1 mm i.d.×150 mm TSKGel G5000 or G6000 columns in series (Tosoh Bioscience, Japan), isocratic flow at 25 μl/min and a 30 min run time. The GPC buffer used is 5 mM ammonium acetate in water containing 10% acetonitrile. For mass spectrometric analysis, the first quadruple is operated at the wide band RF only mode so all ions are passed through the quadrupole, fragmented at an elevated collision energy, e.g., CE 70V in the 2nd quadrupole, and analyzed by the TOF mass analyzer. The mass range scanned is typically 30 to 3000 Da.

### Data Acquisition, Processing and Analysis

The QTOF mass spectrometer uses MassLynx (Waters) for data acquisition and data processing. The peak intensity of the major common fragment ion, e.g., a water soluble material with a poly(vinyl alcohol) backbone at m/z 131 using CE 70V, is measured and averaged from the two replicate CapGPC-QTOF runs. The polymer weight percent concentration in each sample is calculated against the water soluble material standard calibration curve. A good linearity covering is obtained under the current measurement conditions.

Bounded water soluble material level is calculated by using the following equation:

RP=TP−FP

wherein
RP is the percentage of bounded water soluble material in the delivery particle polymer wall,
TP is the percentage of total water soluble material added to the aqueous phase, and
FP is the sum of the free amount of water soluble material in all the aqueous phases collected and measured after delivery particles are made.

### Method for Treating Fabrics With Fabric Softener/Liquid Laundry Detergent Composition Comprising a Population of Delivery Particles Prior to Head Space Concentration Determination

The method to treat fabrics with fabric softener composition comprises a fabric pre-treatment phase followed by a fabric treatment phase.

### Fabric Pretreatment Phase

2.9±0.1 kg of ballast fabrics containing cotton, polyester, polycotton, 3 white knitted cotton fabric tracers (from Warwick Equest) and 3 white polyester tracers are washed 4 times with 50 g Non-perfumed Ariel Sensitive (Nordics) at 60° C. with 2 grains per gallon (gpg) water, 1 h 26 min cycle, 1600 rpm, in a front loader washing machine such as Miele (Novotronic W986/Softronic W467/W526/W527/W1614/W1714/W2261) or equivalent and then washed once with no detergent at 60° C. with 2 gpg water. After the last wash, fabrics are dried in a 5 kg drum tumble drier with hot air outlet such as Miele Novotronic (T490/T220/T454/T430/T410/T7634) or equivalent and then they are ready to be used for testing.

### Fabric Treatment Phase

2.9±0.1 kg of ballast fabrics containing cotton, polyester, polycotton, 3 white knitted cotton fabric tracers (from Warwick Equest) and 3 white polyester tracers are washed in 15 gpg water under different conditions depending on the product to be tested:
1. at 40° C., 1 h 24 minutes cycle, 1200 rpm without laundry detergent to avoid interference in the fabric softener evaluation. Liquid fabric softener composition is pre-diluted in 2 L of 15° C. water with a hardness of 15 gpg 5 min before the starting of the last rinse and added to the last rinse while the washing machine is taking the water. This is a requirement to ensure homogeneous dispensability over the load and minimize the variability of the test results. All fabrics are line dried in a control temperature (25° C.) and humidity (60%) room for 24 hours prior to head space concentration determination; or
2. at 30° C., 1 h 15 minutes cycle, 1000 rpm using the laundry detergent to be evaluated without fabric softener. The laundry detergent is dosed in a dosing ball and introduced in the tumble together with the fabrics.

### Method for Determining Head Space Concentration

Three white knitted cotton fabric tracers and/or 3 white polyester fabric tracers treated with fabric softener compositions (see Method 3 for treating fabrics with fabric softener composition prior to head space concentration determination) are used for the analysis. A piece of 5×5 cm is gently cut from the center of each fabric tracer and analyzed by fast head space gas chromatography/mass spectroscopy (“GC/MS”) using an Agilent DB-5UI 30 m×0.25×0.25 column (part #122-5532UI) in splitless mode. Each fabric tracer cut is transferred into 25 mL glass headspace vials. The fabric samples are allowed to equilibrate for 10 minutes at 65° C. before the headspace above the fabrics is sampled using a 23 gauge 50/30UM DVB/CAR/PDMS SPME fiber (Sigma-Aldrich part #57298-U) for 5 minutes. The SPME fiber is subsequently on-line thermally desorbed into the GC using a ramp from 40° C. (0.5 min) to 270° C. (0.25 min) at 17° C./min. The perfume raw materials with a molecular weight between 35 and 300 m/z are analyzed by fast GC/MS in full scan mode. The amount of perfume in the headspace is expressed as nmol/L.

### Sample Preparation for Biodegradability Measurements

The water soluble material is purified via crystallization till a purity of above 95% is achieved and dried before biodegradability measurement.

The core needs to be extracted from the delivery particle slurry in order to only analyze the polymer wall. Therefore, the delivery particle slurry is washed between 3 and 10 times with water to remove all materials that are not reacted in the polymer wall, such as free water soluble material, colloids and depositions aids. Then, it is further washed with organic solvents to extract the core till weight percentage of core is below 5% based on total delivery particle polymer wall weight. Finally, the polymer wall is dried and analyzed.

Weight ratio of delivery particle:solvent is 1:3. Residual core is determined by thermogravimetric analysis (60 minutes isotherm at 100° C. and another 60 min isotherm at 250° C.). The weight loss corresponding to the core determined needs to be below 5%.

### OECD 301 B—Biodegradability Method

Accumulative CO₂ release is measured over 60 days following the guidelines of the Organisation for Economic Cooperation and Development (OECD)—OECD (1992), Test No. 301: Ready Biodegradability, OECD Guidelines for the Testing of Chemicals, Section 3, OECD Publishing, Paris, https://doi.org/10.1787/9789264070349-en.

### Extraction of Delivery Particles From Consumer Products

Except where otherwise specified herein, the preferred method to isolate delivery particles from consumer products is based on differing densities, in that the density of most such particles is different from that of the consumer product. The consumer product is mixed with water in order to dilute and/or release the particles. The diluted product suspension is centrifuged to speed up the separation of the particles. Such particles tend to float or sink in the diluted solution/dispersion of the consumer product depending on its density. Using a pipette or spatula, the top and/or bottom layers of this suspension are removed and undergo further rounds of dilution and centrifugation to separate and enrich the particles. The particles are observed using an optical microscope equipped with crossed-polarized filters or differential interference contrast (DIC), at total magnifications of 100× and 400×. The microscopic observations provide an initial indication of the presence, size, quality and aggregation of the delivery particles.

For extraction of delivery particles from a liquid consumer product such as liquid fabric enhancer, liquid laundry detergent, shampoo, and/or hair treatment composition, conduct the following procedure:
1. Place three aliquots of approximately 20 ml of liquid consumer product comprising the delivery particles into three separate 50 ml centrifuge tubes and dilute each aliquot 1:1 with DI water (e.g. 20 ml fabric enhancer+20 ml DI water), mix each aliquot well and centrifuge each aliquot for 30 minutes at approximately 4,000 rpm. If necessary, NaCl (e.g., 100-200 mg NaCl) can be added to the diluted suspension in order to increase the density of the solution and facilitate the particles floating to the top layer.
2. After centrifuging per Step 1, discard the bottom water layer (around 10 ml) in each 50 ml centrifuge tube then add 10 ml of DI water to each 50 ml centrifuge tube.
3. For each aliquot, repeat the process of centrifuging, removing the bottom water layer and then adding 10 ml of DI water to each 50 ml centrifuge tube two additional times.
4. Remove the top layer with a spatula or a pipette.
5. Transfer this top layer into a 1.8 ml centrifuge tube and centrifuge for 5 minutes at approximately 14,000 rpm.
6. Remove the top layer with a spatula and transfer into a new 1.8 ml centrifuge tube and add DI water until the tube is completely filled, then centrifuge for 5 minutes at approximately 14,000 rpm.
7. Remove the bottom layer with a fine pipette and add DI water until tube is filled and centrifuge for 5 minutes at approximately 14,000 rpm.
8. Repeat step 7 for an additional 5 times (6 times in total).

If both a top layer and a bottom layer of enriched delivery particles appear in the above described step 1, then, immediately move to step 3 (i.e., omit step 2) and proceed steps with steps 4 through 8. Once those steps have been completed, also remove the bottom layer from the 50 ml centrifuge tube from step 1, using a spatula or/and a pipette. Transfer the bottom layer into a 1.8 ml centrifuge tube and centrifuge 5 min at approximately 14,000 rpm. Remove the bottom layer in a new tube and add DI water until the tube is completely filled then centrifuge for 5 minutes approximately 14,000 rpm. Remove the top layer (water) and add DI water again until the tube is full. Repeat this another 5 times (6 times in total). Recombine the particle enriched and isolated top and bottom layers back together.

If the liquid consumer product has a white color or is difficult to distinguish the particle enriched layers add 4 drops of dye (such as Liquitint Blue JH 5% premix from Milliken & Company, Spartanburg, S.C., USA) into the centrifuge tube of step 1 and proceed with the isolation as described.

For extraction of delivery particles from solid consumer products which disperse readily in water, mix 1 L of DI water with 20 g of the consumer product containing the delivery particles (e.g. detergent foams, films, gels and granules; or water-soluble polymers; soap flakes and soap bars; and other readily water-soluble matrices such as salts, sugars, clays, and starches). When extracting particles from finished products which do not disperse readily in water, such as waxes, dryer sheets, dryer bars, and greasy materials, it may be necessary to add detergents, agitation, and/or gently heat the product and diluent in order to release the particles from the matrix. The use of organic solvents or drying out of the particles should be avoided during the extraction steps as these actions may damage the delivery particles during this phase.

One skilled in the art will recognize that various other protocols may be constructed for the extraction and isolation of delivery particles from finished products, and will recognize that such methods require validation via a comparison of the resulting measured values, as measured before and after the particles' addition to and extraction from finished product.

### Leakage

The amount of benefit agent leakage from the benefit agent containing delivery particles is determined according to the following method:

xxx
1. Obtain two 1 g samples of the delivery particles slurry.
2. Add 1 g of the delivery particles slurry to 99 g of the consumer product matrix in which the delivery particles will be employed and label the mixture as Sample 1. Immediately use the second 1 g sample of delivery particles slurry in Step d below, in its neat form without contacting consumer product matrix, and label it as Sample 2.
3. Age the delivery particle-containing product matrix (Sample 1) for 1 week at 35° C. in a sealed glass jar.
4. Using filtration, recover the particles from both samples. The particles in Sample 1 (in consumer product matrix) are recovered after the aging step. The particles in Sample 2 (neat raw material slurry) are recovered at the same time that the aging step began for sample 1.
5. Treat the recovered particles with a solvent to extract the benefit agent materials from the particles.
6. Analyze the solvent containing the extracted benefit agent from each sample, via chromatography.
7. Integrate the resultant benefit agent peak areas under the curve and sum these areas to determine the total quantity of benefit agent extracted from each sample.
8. Determine the percentage of benefit agent leakage by calculating the difference in the values obtained for the total quantity of benefit agent extracted from Sample 2 (S2) minus Sample 1 (S1), expressed as a percentage of the total quantity of benefit agent extracted from Sample 2 (s2), as represented in the equation below:

$$\% Leakage = ( \frac{S 2 - S 1}{S 2} ) \times 100$$

### Volume Weighted Mean Particle Size

Particle size is measured using static light scattering devices, such as an Accusizer 780A, made by Particle Sizing Systems, Santa Barbara Calif. The instrument is calibrated from 0 to 300 μusing Duke particle size standards. Samples for particle size evaluation are prepared by diluting about 1 g emulsion, if the volume weighted mean particle size of the emulsion is to be determined, or 1 g of benefit agent containing delivery particles slurry, if the finished particles volume weighted mean particle size is to be determined, in about 5 g of de-ionized water and further diluting about 1 g of this solution in about 25 g of water.

About 1 g of the most dilute sample is added to the Accusizer and the testing initiated, using the autodilution feature. The Accusizer should be reading in excess of 9200 counts/second. If the counts are less than 9200 additional sample should be added. The Accusizer will dilute the test sample until 9200 counts/second and initiate the evaluation. After 2 minutes of testing the Accusizer will display the results, including volume-weighted mean size.

The broadness index can be calculated by determining the particle size at which 95% of the cumulative particle volume is exceeded (95% size), the particle size at which 5% of the cumulative particle volume is exceeded (5% size), and the mean volume-weighted particle size (50% size−50% of the particle volume both above and below this size). Broadness Index (5)=((95% size)−(5% size)/50% size).

### Gel Permeation Chromatography With Multi-Angle Light Scattering and Refractive Index Detection (GPC-MALS/RI) for Polymer Molecular Weight Distribution Measurement

Gel Permeation Chromatography (GPC) with Multi-Angle Light Scattering (MALS) and Refractive Index (RI) Detection (GPC-MALS/RI) permits the measurement of absolute molecular weight of a polymer or water soluble material without the need for column calibration methods or standards. The GPC system allows molecules to be separated as a function of their molecular size. MALS and RI allow information to be obtained on the number average (Mn) and weight average (Mw) molecular weight. The Mw distribution of water-soluble polymers like polyvinylalcohol, polysaccharides, polyacrylates materials is typically measured by using a Liquid Chromatography system (e.g., Agilent 1260 Infinity pump system with OpenLab Chemstation software, Agilent Technology, Santa Clara, CA, USA) and a column set (e.g., Waters ultrahydrogel guard column, 6 mm ID×40 mm length, two ultrahydrogel linear columns, 7.8 mm ID×300 mm length, Waters Corporation of Milford, Mass., USA) which is operated at 40° C. The mobile phase is 0.1M sodium nitrate in water containing 0.02% sodium azide and is pumped at a flow rate of 1 mL/min, isocratically. A multiangle light scattering (MALS) detector DAWN® and a differential refractive index (RI) detector (Wyatt Technology of Santa Barbara, Calif, USA) controlled by Wyatt Astra® software are used. A sample is typically prepared by dissolving polymer materials in the mobile phase at ˜1 mg per ml and by mixing the solution for overnight hydration at room temperature. The sample is filtered through a 0.8 μm Versapor membrane filter (PALL, Life Sciences, NY, USA) into the LC autosampler vial using a 3-ml syringe before the GPC analysis. A dn/dc (differential change of refractive index with concentration) value is measured on the polymer materials of interest and used for the number average and weight average molecular weights determination by the Astra detector software.

### Percent Solids Test Method

The percent solids of a sample, such as capsule slurries (100% minus the percent weight fraction of water of a sample), is determined by volatilizing the water of the sample via heat and ventilation, while controlling temperature of the sample, to prevent and minimize volatilization of non-water components in the sample, measuring the weight of the sample before and after water volatilization, once the weight of the sample has leveled off to a small enough tolerance. The following method was used:

Using a moisture/solids analyzer, such as a CEM Smart Turbo (CEM Corporation, Mathews NC), with drying pads, such as CEM 4″×4″ glass fiber drying pads, 1-2 g of sample at room temperature placed and spread between two pads, pads then placed on a microbalance within 60 seconds of applying sample to pads.

Solids program run until completion, where the program is set to regulate the sample temperature at 50° C., volatilizing until the weight loss is less than 0.5 mg in 10 seconds.

### EXAMPLES

### Example 1: Preparation of Water Soluble Material Based on Poly(Vinyl Alcohol) Backbone Polymer (Mod-PVOH)

Samples 1-5, as shown below in TABLE 1, are prepared in a 2 L 3-necked-flask, methacrylic anhydride (Merck) is added to a mixture comprising Selvol 540 (Sekisui Specialty Chemicals), Dimethylacetamide—DMAC—(Merck), pyridine (Merck) and Butylhydroxytoluene—BHT—(Merck). Then the mixture is heated under magnetic stirring at 50° C. for 18 hours. Product is cooled to 20° C. and 1000 g of demineralized water are added, then the mixture is filtered to remove insoluble materials. Acetone (6272 g Merck) is then added to precipitate the mod-PVOH. In order to fully remove DMAC, 4 cycles of decantation and acetone (3130 g each) washing are applied to obtain a powdery polymer, which is dried under high vacuum. The precipitate is stirred for 24 hours in acetone and then further dried at 50° C. in a vacuum oven (Lab Companion Vacuum Oven from Cole-Parmer). Finally, mod-PVOH is characterized by ¹H-NMR, as described in Method 1.

TABLE 1

| | Weight (g) | | | | |
|---|---|---|---|---|---|
| | Mod- | Mod- | Mod- | Mod- | Mod- |
| | PVOH | PVOH | PVOH | PVOH | PVOH |
| | Sam- | Sam- | Sam- | Sam- | Sam- |
| | ple 1 | ple 2 | ple 3 | ple 4 | ple 5 |
| Methacrylic | 4.14 | 10.36 | 18.65 | 26.9 | 35.22 |
| anhydride | | | | | |
| Selvol 540 | 75 | | | | |
| DMAC | 937 | | | | |
| Pyridine | 5.87 | 14.66 | 26.31 | 38.06 | 49.68 |
| BHT | 0.2292 | 0.7495 | 1.3408 | 1.9230 | 2.5174 |
| Degree of | 1 | 3 | 5 | 7 | 9 |
| substitution (%) | | | | | |

### Example 2: Delivery Particles

### Sample 6 Delivery Particles

A first oil phase was prepared by mixing 63.1 g perfume oil, 6.2 g CN975, 0.076 g TBAEMA, and 0.076 g CD9055 until a homogenous mixture was obtained.

A second oil phase was prepared by mixing 143.1 g of the perfume oil, 137.5 g isopropyl myristate, and 0.338 g 2,2′-azobis(2-methylbutyronitrile) in a jacketed steel reactor. The reactor was held at 35° C. and the oil solution mixed. A nitrogen blanket was applied to the reactor at a rate of 100 cc/min. The second oil composition was heated to 70° C. in 45 minutes, held at 70° C. for 45 minutes, then cooled to 50° C. in 45 minutes. Once cooled to 50° C. the first oil phase was added, and the combined oils mixed for another 10 minutes at 50° C.

A water phase was prepared by dissolving 5.36 g of modified poly(vinyl alcohol) from Sample 1 in 441.6 g demineralized water for 16 hours at 20° C. Then, 0.225 g 4,4′-azobis[4-cyanovaleric acid] and 0.211 g of a 21.5% aqueous solution of sodium hydroxide were added to the water phase and mixed until homogenous. Water phase was heated to 50° C. within an hour prior and then transferred to the mixed first and second oil phases.

High shear agitation was then applied over 60 minutes to produce an emulsion with volume weighted mean size of 30.97 μm, determined via Method 9. The reactor was then mixed with a 3″ diameter marine propeller blade, 100 g demineralized water added, covered, and the temperature increased to 75° C. in 60 minutes, held at 75° C. for 4 hours, increased to 95° C. in 60 minutes, and held at 95° C. for 6 hours. The batch was cooled to 25° C. within a couple hours. The percentage of solids was measured, and demineralized water added to adjust solids back to 44.56 wt % solids to account for water evaporation during reaction. The amount of free and bounded water soluble material is determined via Method 2, being 91.1% wt the amount of bounded water soluble material.

### Sample 7 Delivery Particles

A first oil phase was prepared by mixing 35.5 g perfume oil, 3.5 g CN975, 0.043 g TBAEMA, and 0.043 g CD9055 until a homogenous mixture was obtained.

A second oil phase was prepared by mixing 80.5 g of the perfume oil, 77.3 g isopropyl myristate, and 0.19 g 2,2′-azobis(2-methylbutyronitrile) in a jacketed steel reactor. The reactor was held at 35° C. and the oil solution mixed. A nitrogen blanket was applied to the reactor at a rate of 100 cc/min. The second oil composition was heated to 70° C. in 45 minutes, held at 70° C. for 45 minutes, then cooled to 50° C. in 45 minutes. Once cooled to 50° C. the first oil phase was added, and the combined oils mixed for another 10 minutes at 50° C.

A water phase was prepared by dissolving 2.97 g of modified poly(vinyl alcohol) from Sample 2 in 189.5 g demineralized water for 16 hours at 20° C. Then, 60.2 g of a 5 wt % aqueous solution of Selvol 540, 0.127 g 4,4′-azobis[4-cyanovaleric acid] and 0.119 g of a 21.5% aqueous solution of sodium hydroxide added to the water phase and mixed until homogenous. Water phase was heated to 50° C. within an hour prior and then transferred to the mixed first and second oil phases.

High shear agitation was applied over 60 minutes to produce an emulsion with volume weighted mean size of 33.5 μm, determined via Method 9. The reactor was then mixed with a 3″ diameter marine propeller blade, 100 g demineralized water added, covered, and the temperature increased to 75° C. in 60 minutes, held at 75° C. for 4 hours, increased to 95° C. in 60 minutes, and held at 95° C. for 6 hours. The batch was cooled to 25° C. within a couple hours. The percentage of solids was measured, and demineralized water added to adjust solids back to 45.08 wt % solids to account for water evaporation during reaction.

### Sample 8 Delivery Particles

A water phase was prepared by mixing 174.6 g demineralized water and 59 g of a 5 wt % aqueous solution of Selvol 540. Then, 16.4 g of a 5 wt % aqueous solution of anhydrous sodium carbonate and 0.59 g methacrylic anhydride were added. After mixing for 16 hours, 1.39 g 20 wt % aqueous solution of hydrochloric acid was added. Then 0.127 g 4,4′-azobis[4-cyanovaleric acid] and 0.119 g of a 21.5% aqueous solution of sodium hydroxide were added and mixed until homogenous. The water phase heated to 50° C. within an hour prior to transferring in subsequent steps.

A first oil phase was prepared by mixing 35.5 g perfume oil, 3.5 g CN975, 0.043 g TBAEMA, and 0.043 g CD9055 until a homogenous mixture was obtained.

A second oil phase was prepared by mixing 80.5 g of the perfume oil, 77.3 g isopropyl myristate, and 0.19 g 2,2′-azobis(2-methylbutyronitrile) in a jacketed steel reactor. The reactor was held at 35° C. and the oil solution mixed. A nitrogen blanket was applied to the reactor at a rate of 100 cc/min. The second oil composition was heated to 70° C. in 45 minutes, held at 70° C. for 45 minutes, then cooled to 50° C. in 45 minutes. Once cooled to 50° C. the first oil phase was added, and the combined oils mixed for another 10 minutes at 50° C.

The water phase was transferred to the mixed first and second oil phases. High shear agitation was then applied over 60 minutes to produce an emulsion with volume weighted mean size of 34.69 μm, determined via Method 9. The reactor was then mixed with a 3″ diameter marine propeller blade, 100 g demineralized water added, covered, and the temperature increased to 75° C. in 60 minutes, held at 75° C. for 4 hours, increased to 95° C. in 60 minutes, and held at 95° C. for 6 hours. The batch was cooled to 25° C. within a couple hours. The percentage of solids was measured, and demineralized water added to adjust solids back to 44.93 wt % solids to account for water evaporation during reaction. The amount of free and bounded water soluble material is determined via Method 2, being 69.9% wt the amount of bounded water soluble material.

### Sample 9 Delivery Particles

A water phase was prepared by mixing 157.5 g demineralized water and 58.5 g of a 5 wt % aqueous solution of Selvol 540. Then, 32.1 g of a 5 wt % aqueous solution of anhydrous sodium carbonate and 1.17 g methacrylic anhydride added. After mixing for 16 hours, 2.76 g 20 wt % aqueous solution of hydrochloric acid were added. Then, 0.127 g 4,4′-azobis[4-cyanovaleric acid] and 0.119 g of a 21.5% aqueous solution of sodium hydroxide were added and mixed until homogenous. Water phase was heated to 50° C. within an hour prior to transferring in subsequent steps.

A first oil phase was prepared by mixing 35.5 g perfume oil, 3.5 g CN975, 0.043 g TBAEMA, and 0.043 g CD9055 until a homogenous mixture was obtained.

A second oil phase was prepared by mixing 80.5 g of the perfume oil, 77.3 g isopropyl myristate, and 0.19 g 2,2′-azobis(2-methylbutyronitrile) in a jacketed steel reactor. The reactor was held at 35° C. and the oil solution mixed. A nitrogen blanket was applied to the reactor at a rate of 100 cc/min. The second oil composition was heated to 70° C. in 45 minutes, held at 70° C. for 45 minutes, then cooled to 50° C. in 45 minutes. Once cooled to 50° C. the first oil phase was added, and the combined oils mixed for another 10 minutes at 50° C.

The water phase was transferred to the mixed first and second oil phases. High shear agitation was then applied over 60 minutes to produce an emulsion with volume weighted mean size of 36.34 μm, determined via Method 9. The reactor was then mixed with a 3″ diameter marine propeller blade, 100 g demineralized water added, covered, and the temperature increased to 75° C. in 60 minutes, held at 75° C. for 4 hours, increased to 95° C. in 60 minutes, and held at 95° C. for 6 hours. The batch was cooled to 25° C. within a couple hours. The percentage of solids was measured, and demineralized water added to adjust solids back to 46.24% wt % solids to account for water evaporation during reaction. The amount of free and bounded water soluble material is determined via Method 2, being 74.6% wt the amount of bounded water soluble material.

### Example 3: Liquid Fabric Softener Comprising Delivery Particles

Liquid Fabrice Softener (Samples 6A-9A) comprising Delivery Particles (Samples 6-9) was prepared as described below, and the Delivery Particles tested for leakage, as described in the TEST METHODS Section, as shown in TABLE 2.

A fabric softener composition was prepared according to WO2018/170356. The fabric softener composition (Samples 6A-9A) was finished by adding the delivery particle slurry (Samples 6-9) using an IKA Ultra Turrax (dispersing element 8G) operated at 10 000 rpm for 1 minute, as shown below in TABLE 2.

TABLE 2

| | | | |
|---|---|---|---|
| Sam- | Sam- | Sam- | Sam- |
| ple 6A | ple 7A | ple 8A | ple 9A |

Weight %

| | | | | |
|---|---|---|---|---|
| Deionized water | To | To | To | To |
| | balance | balance | balance | balance |
| NaHEDP | 0.007 | 0.007 | 0.007 | 0.007 |
| Formic acid | 0.045 | 0.045 | 0.045 | 0.045 |
| HCl | 0.001 | 0.001 | 0.001 | 0.001 |
| Preservative^{a} | 0.023 | 0.023 | 0.023 | 0.023 |
| FSA^{b} | 9.19 | 9.19 | 9.19 | 9.19 |
| Antifoam^{c} | 0.101 | 0.101 | 0.101 | 0.101 |
| Coconut oil | 0.31 | 0.31 | 0.31 | 0.31 |
| Isopropanol | 0.94 | 0.94 | 0.94 | 0.94 |
| CaCl₂ | 0.008 | 0.008 | 0.008 | 0.008 |
| Perfume | 0.4 | | | |
| Perfume via | 0.25 | | | |
| delivery particles | | | | |
| from Sample 6 | | | | |
| Perfume via | | 0.4 | | |
| delivery particles | | | | |
| from Sample 7 | | | | |
| Perfume via | | | 0.4 | |
| delivery particles | | | | |
| from Sample 8 | | | | |
| Perfume via | | | | 0.4 |
| delivery particles | | | | |
| from Sample 9 | | | | |
| Cationic | 0.3 | 0.3 | 0.3 | 0.3 |
| polymer^{d} | | | | |
| Leakage (Method | | 15.88% | 9.38% | 10.02% |
| 8) | | | | |
| Performance^{e} | | | | |

^{a}Proxel GXL, 20% aqueous dipropylene glycol solution of 1,2-benzisothiazolin-3-one, supplied by Lonza. This material is part of the dispersion that is made and is not added at another point in the process.
^{b}DEEDMAC: diethyl-ester-dimethyl-ammonium-chloride
^{c}MP10 ®, supplied by Dow Corning, 8% activity
^{d}Rheovis ® CDE, cationic polymeric acrylate thickener supplied by BASF
^{e}Method 3 is used for the preparation of the fabrics and Method 4 to measure the amount of perfume released

TABLE 2 demonstrates a liquid fabric softener composition comprising biodegradable delivery particles with low leakage and delivery perfume in the wash.

### Example 4: Liquid Laundry Detergent Comprising Delivery Particles

Liquid Laundry Detergent Compositions (Samples 6B-9B and Samples 6C-9C) comprising the Delivery Particles of Samples 6-9 were prepared and the Delivery Particles tested for leakage, as described in the TEST METHODS Section, and shown in TABLES 3 and 4 below.

TABLE 3

| | Sam- | Sam- | Sam- | Sam- |
|---|---|---|---|---|
| | ple 6B | ple 7B | ple 8B | ple 9B |
| Ingredient: | % wt | | | |
| C12-45 alkyl-7-ethoxylated | 2.34 | | | |
| C12-14 alkyl-7-ethoxylated | 0.2 | | | |
| Monoethanolamine: C₁₂₋₁₄ | 0.5 | | | |
| EO•3•SO₃H | | | | |
| Linear alkyl benzene sulfonic acid | 4 | | | |
| sodium hydroxide | 1.9 | | | |
| sodium cumene sulfonate | 0.18 | | | |
| citric acid | 1.4 | | | |
| C12-18 Fatty acid | 1.1 | | | |
| Solvents (1,2-Propanediol, | 1.1 | | | |
| Ethanol) | | | | |
| Chelants | 0.2 | | | |
| Soil suspending alkoxylated | 0.68 | | | |
| polyalkylenimine polymer^{a} | | | | |
| Minors (stabilizers, | 1 | | | |
| preservatives . . .) | | | | |
| Hydrogenated castor oil | 0.2 | | | |
| Perfume via delivery particles from | 0.5 | | | |
| Sample 6 | | | | |
| Perfume via delivery particles from | | 0.5 | | |
| Sample 7 | | | | |
| Perfume via delivery particles from | | | 0.5 | |
| Sample 8 | | | | |
| Perfume via delivery particles from | | | | 0.5 |
| Sample 9 | | | | |
| water | up to 100 | | | |
| Leakage | 20.8 | 17.2 | 5 | 5.3 |

^{a}600 g/mol molecular weight polyethylenimine core with 24 ethoxylate groups per —NH and 16 propoxylate groups per —NH. Available from BASF (Ludwigshafen, Germany)

TABLE 3 demonstrates a liquid laundry detergent composition comprising biodegradable delivery particles with low leakage.

TABLE 4

| | Example: | | | |
|---|---|---|---|---|
| | Sam- | Sam- | Sam- | Sam- |
| | ple 6C | ple 7C | ple 8C | ple 9C |
| Ingredient: | % wt | | | |
| Alkyl ether sulfate | 3.96 | | | |
| Linear alkyl benzene sulfonic acid | 9.15 | | | |
| Ethoxylated alcohol | 3.83 | | | |
| Amine oxide | 0.51 | | | |
| Fatty acid | 1.73 | | | |
| citric acid | 2.79 | | | |
| Sodium diethylene triamine penta | 0.512 | | | |
| methylene phosphonic acid | | | | |
| Calcium chloride | 0.011 | | | |
| Sodium formate | 0.034 | | | |
| Ethoxysulfated hexamethylene | 0.664 | | | |
| diamine quaternized | | | | |
| Co-polymer of Polyethylene glycol | 1.27 | | | |
| and vinyl acetate | | | | |
| Optical Brightener 49 | 0.046 | | | |
| 1,2-benzisothiazolin-3-one and 2- | 0.005 | | | |
| methyl-4-isothiazolin-3-one | | | | |
| Ethanol | 0.42 | | | |
| 1,2-propanediol | 1.26 | | | |
| sodium cumene sulfonate | 1.72 | | | |
| Mono ethanol amine | 0.24 | | | |
| Sodium hydroxide | 3.1 | | | |
| Hydrogenated castor oil | 0.3 | | | |
| Silicone emulsion | 0.0025 | | | |
| Dye | 0.0054 | | | |
| Minors (stabilizers, | 1 | | | |
| preservatives . . .) | | | | |
| Hydrogenated castor oil | 0.2 | | | |
| Perfume | 0.6 | | | |
| Perfume via delivery particles from | 0.4 | | | |
| Sample 6 | | | | |
| Perfume via delivery particles from | | 0.25 | | |
| Sample 7 | | | | |
| Perfume via delivery particles from | | | 0.1 | |
| Sample 8 | | | | |
| Perfume via delivery particles from | | | | 0.2 |
| Sample 9 | | | | |

| | |
|---|---|
| water | up to 100 |

### Example 5: Shampoo Compositions Comprising Delivery Particles

Shampoo Compositions (Samples 6D-9D) comprising the Delivery Particles of Samples 6-9 were prepared and the Delivery Particles tested for leakage, as described in the TEST METHODS Section, and shown in TABLE 5 below.

TABLE 5

| | Sam- | Sam- | Sam- | Sam- |
|---|---|---|---|---|
| | ple 6D | ple 7D | ple 8D | ple 9D |
| Ingredient: | % wt | | | |
| Sodium laureth sulfate | 14.5 | | | |
| Cocamidopropyl betaine | 1.7 | | | |
| Ethylenediaminetetraacetic acid | 0.15 | | | |
| tetrasodium salt | | | | |
| Sodium benzoate | 0.25 | | | |
| Glycerin | 0.5 | | | |
| Guar Hydroxypropyltrimonium | 0.33 | | | |
| Chloride^{a} | | | | |
| Polyquaternium-10^{b} | 0.075 | | | |
| Polyquarternium-6^{c} | 0.075 | | | |
| Cetyl alcohol | 0.675 | | | |
| Stearyl alcohol | 1.075 | | | |
| Dimethicone^{d} | 0.8 | | | |
| 5-chloro-2-methyl-4-isothiazolin-3- | 0.033 | | | |
| one, Kathon CG | | | | |
| Acrylate copolymer^{e} | | 1.5 | | 2 |
| Hydrogenated castor oil | | | 0.5 | |
| Argan oil | | 2 | | |
| Menthol | | | 2 | |
| Panthenol | | | | 1.5 |
| Perfume | | 1 | 1 | 0.5 |
| Perfume via delivery particles from | 0.5 | | | |
| Sample 6 | | | | |
| Perfume via delivery particles from | | 0.5 | | |
| Sample 7 | | | | |
| Perfume via delivery particles from | | | 0.25 | |
| Sample 8 | | | | |
| Perfume via delivery particles from | | | | 0.25 |
| Sample 9 | | | | |
| water | up to 100 | | | |
| Leakage | 13.26% | | | |

^{a}N-Hance 3196 from Ashland
^{b}Polymer KG30M from Dow Chemicals
^{c}Flocare C 106 from SNF
^{d}Belsil DM 5500E from Wacker
^{e}Carbopol Aqua SF 1 from Lubrizol

The Results of TABLE 5 demonstrate that biodegradable delivery particles have a low leakage in shampoo.

### Example 6: Leave-On Treatment Comprising Delivery Particles

Leave-On Treatment Compositions (Samples 6E-9E) comprising the Delivery Particles of Samples 6-9 were prepared and the Delivery Particles tested for leakage, as described in the TEST METHODS Section, and shown in TABLE 6 below.

TABLE 6

| | Sam- | Sam- | Sam- | Sam- |
|---|---|---|---|---|
| | ple 6E | ple 7E | ple 8E | ple 9E |
| Ingredient: | % wt | | | |
| Polyacrylamide & C13-14 | 0.82 | | | |
| isoparaffin & Laureth-7 & AQUA | | | | |
| Phenoxyethanol and ethylhexyl | 1 | | | |
| glycerin | | | | |
| Benzyl alcohol | 0.4 | | | |
| Perfume | | 0.3 | 0.5 | 0.2 |
| Perfume via delivery particles from | 0.25 | | | |
| Example 6 | | | | |
| Perfume via delivery particles from | | 0.5 | | |
| Example 7 | | | | |
| Perfume via delivery particles from | | | 0.25 | |
| Example 8 | | | | |
| Perfume via delivery particles from | | | | 0.25 |
| Example 9 | | | | |
| water | up to 100 | | | |
| Leakage | 33.5% | | | |

The results of TABLE 6 demonstrate that biodegradable delivery particles have a low leakage in leave-on treatments

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A delivery particle comprising a core and a polymer wall encapsulating said core, wherein:
the core comprises a benefit agent and a partitioning modifier;
the polymer wall is formed by cross-linking of
i. at least one water soluble material that has following Formula I 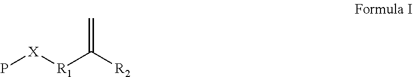
wherein
P is a polymer backbone with a molecular weight of from about 30 kDa to about 500 kDa, selected from the group consisting of poly(vinyl alcohol), chitosan, chitin, pectin, carrageenan, xanthan gum, tara gum, konjac gum, alginate, hyaluronic acid, amylose, lignin, diutan gum and mixtures thereof;
X is a polymer heteroatom functionalized with a radical polymerizable group, being preferably O or N;
R₁ is independently selected from the group consisting of 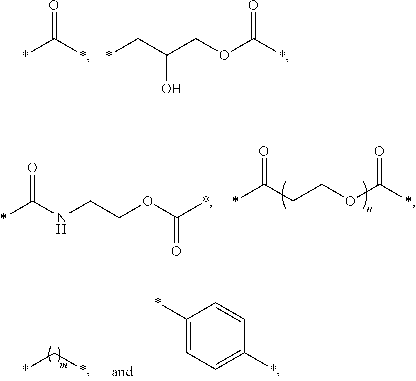
wherein m and n are integers independently selected from 1 to 100, preferably from 1 to 50, even more preferably from 1 to 10;
R₂ independently selected from the group consisting of —H and/or —CH₃;
and being at least 20%, preferably 30%, even more preferably 40% (max 95%) weight percentage of the total wall polymer;
ii. at least one multi-functional monomer and/or oligomer comprising a radical polymerizable group, being at least 5 wt % of the overall polymer wall;
iii. water soluble initiators, oil soluble initiators and mixtures thereof;
iv. optionally, a mono-functional (meth)acrylate monomer.

2. The delivery particle of claim 1, wherein the benefit agent is a fragrance, preferably a fragrance comprising perfume raw materials characterized by a logP of from about 2.5 to about 4.5.

3. The delivery particle of claim 1, wherein the partitioning modifier is selected from the group consisting of isopropyl myristate, vegetable oil, modified vegetable oil, mono-, di-, and tri-esters of C₄-C₂₄ fatty acids, dodecanophenone, lauryl laurate, methyl behenate, methyl laurate, methyl palmitate, methyl stearate, and mixtures thereof, preferably isopropyl myristate.

4. The delivery particle of claim 1, wherein P is preferably a polyvinyl alcohol polymer backbone, a chitosan polymer backbone, a chitin polymer backbone or mixtures thereof.

5. The delivery particle of claim 1, wherein the water-soluble material is covalently bonded into the polymer wall.

6. The delivery particle of claim 1, wherein R₁ is preferably 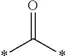 [SMILES: *C(*)=O]
and R₂ a hydrogen or methyl group.

7. The delivery particle of claim 1, wherein the water soluble material is at least 50% wt, preferably at least 75% wt, more preferably at least 85% wt, even more preferably 95% wt of the total polymer wall.

8. The delivery particle of claim 1, wherein the water soluble material has a percentage of functionalized heteroatoms from 0.05 to 20% wt, preferably from 0.5 to 10% wt, even more preferred from 0.6 to 5% wt.

9. The delivery particle of claim 1, wherein the water soluble material comprises at least one (meth)acrylate reactive moiety.

10. The delivery particle of claim 1, wherein the water soluble material has a biodegradability above 30% CO₂ in 60 days following OECD 301B test, preferably above 40% CO₂, more preferably above 50% CO₂, even more preferably above 60% CO₂ (maximum 100%).

11. The delivery particle of claim 1, wherein the polymer wall has a biodegradability from about 30% to about 100% CO₂ in 60 days following OECD 301B test, preferably from about 40% to about 98% CO₂, more preferably from about 50% to about 95% CO₂, even more preferably from about 60% to about 90% CO₂.

12. The delivery particle of claim 1, wherein the water soluble material has a molecular weight from about 30 kDa to about 500 kDa, preferably from about 50 kDa to about 300 kDa, even more preferably from about 80 kDa to about 200 kDa.

13. The delivery particle of claim 1, wherein the multi-functional monomer and/or oligomer comprises a radical polymerizable group selected from the group consisting of acrylate, methacrylate, styrene, allyl, vinyl and mixtures thereof.

14. The delivery particle of claim 1, wherein the multi-functional monomer and/or oligomer comprises a radical polymerizable group selected from the group consisting of acrylate, methacrylate and mixtures thereof.

15. The delivery particle of claim 1, wherein the multi-functional monomer and/or oligomer has at least 2 radical polymerizable groups, at least 3, preferably at least 4, even more preferable at least 5, even more preferably 6.

16. The delivery particle of claim 1, wherein radical polymerizable groups of the multi-functional monomer and/or oligomer are all the same.

17. The delivery particle of claim 1, wherein the polymer wall comprises a combination of at least two multi-functional monomer and/or oligomer.

18. The delivery particle according to any preceding claim forming a population, wherein the delivery particle population comprises the core and polymer wall present in a weight ratio of from about 90:10 to about 99:1, more preferably from about 92:8 to about 98:2.

19. The delivery particle according to any of the preceding claims, having a leakage of below about 50%, preferably about 30%, as determined by the Leakage Test described in the TEST METHODS Section.

20. A method of treating a surface, wherein the method comprises the step of contacting the surface with a composition comprising a delivery particle according to any preceding claim, optionally in the presence of water.

21. A delivery particle comprising a core and a polymer wall encapsulating said core, wherein:
the core comprises a benefit agent and a partitioning modifier;
the polymer wall is a cross-linked polymer comprising sub-units covalently linked by C—C bonds at designated points of attachment or carbon radical centers wherein
i. at least one subunit having the formula IV 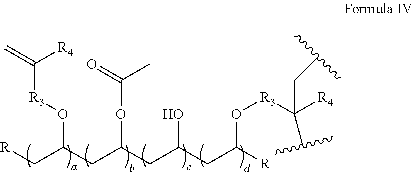
wherein
R₃ is a linking group independently selected from the group consisting of 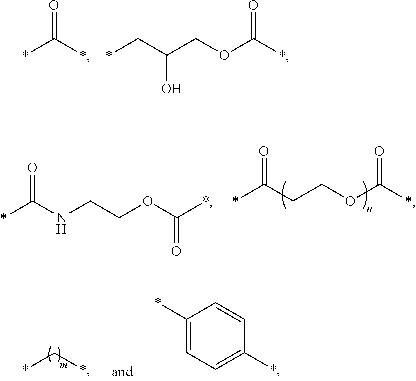
R₄ independently selected from the group consisting of hydrogen and/or methyl;
R is an end group independently selected from the group consisting of vinyl, hydrogen and/or methyl;
a is an integer from 0 to 5000, b, c and d are integers from 1 to 100000, being a+b+c+d at least 340, preferably, a:b:c:d molar ratio is from about 0.0001:98.9:1:0.1 to about 1:59:30:10, preferably from about 0.001:91.8:8:0.2 to about 0.5:70:24.5:5, even more preferably from about 0.001:87:12:1 to about 0.1:79.9:15:5;
ii. at least one subunit having the formula VI; 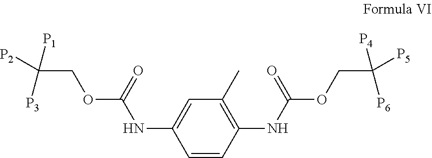
wherein
P₁, P₂, P₃, P₄, P₅ and P₆ can be independently selected from the group consisting of 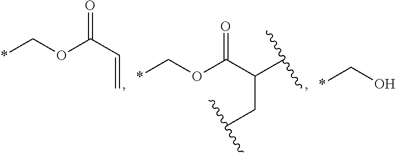
with the proviso that at least three of P₁, P₂, P₃, P₄, P₅ and P₆ are 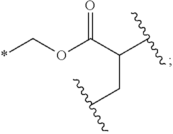 [SMILES: *COC(=O)C(*)C*]
and wherein the subunit of formula VI is at least 5% by weight of the total polymer wall;
iii. at least one subunit having the formula VII and/or VIII 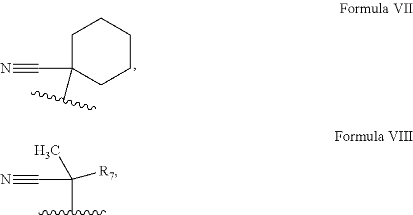
wherein R₇ is independently selected from the group consisting of 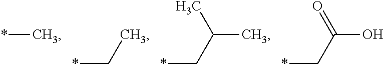
iv. optionally, a subunit having the formula IX and/or X; 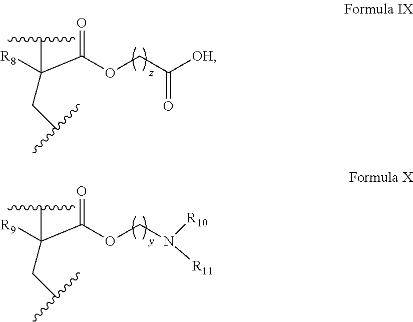
wherein
R₈ and R₉ are independently selected from a hydrogen or a methyl group;
z and y are integers independently selected from 1 to 10, preferably from 2 to 5,
R₁₀ and R₁₁ are independently selected from the group consisting of 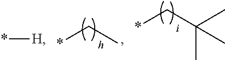
h and i are integers independently selected from 0 to 10, preferably from 1 to 5.

22. A delivery particle comprising a core and a polymer wall encapsulating said core, wherein:
the core comprises a benefit agent and a partitioning modifier;
the polymer wall is a cross-linked polymer comprising sub-units covalently linked by C—C bonds at designated points of attachment or carbon radical centers wherein
i. at least one subunit having the formula V 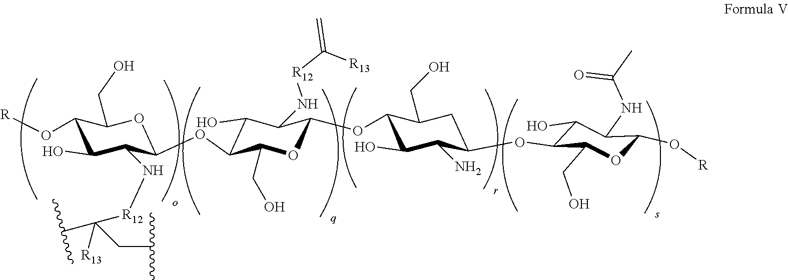
wherein
R₁₂ is a linking group independently selected from the group consisting of 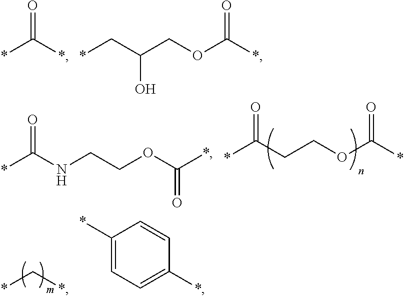
R₁₃ independently selected from the group consisting of hydrogen and/or methyl;
R is an end group independently selected from the group consisting of hydrogen and/or methyl;
q is an integer from 0 to 500, o, r and s are integers from 0 to 100000, being o+q+r+s at least 146, preferably, o:q:r:s molar ratio is from about 0.1:0.0001:98.9:1 to about 10:1:9:80, preferably from about 0.2:0.001:90:9.8 to about 5:0.5:40:54.5, even more preferably from about 1:0.001:87:12 to about 5:0.05:80:14.95;
ii. at least one subunit having the formula VI 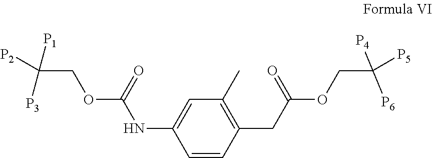
wherein
P₁, P₂, P₃, P₄, P₅ and P₆ can be independently selected from the group consisting of 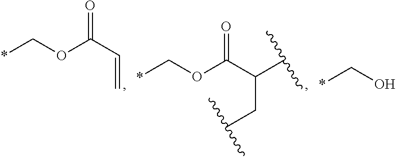
with the proviso that at least three of P₁, P₂, P₃, P₄, P₅ and P₆ are 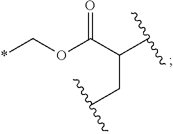 [SMILES: *COC(=O)C(*)C*]
and wherein the subunit of formula VI is at least 5% by weight of the total polymer wall;
v. at least one subunit having the formula VII and/or VIII 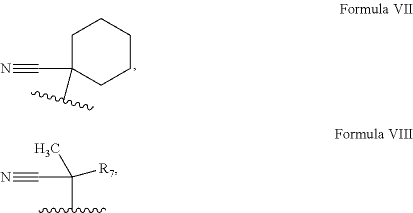
wherein R₇ is independently selected from the group consisting of 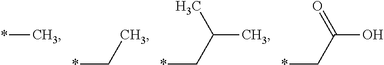
vi. optionally, a subunit having the formula IX or X; 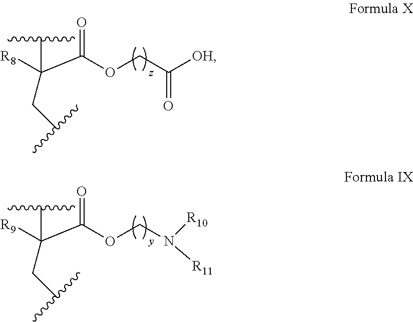
wherein
R₈ and R₉ are independently selected from a hydrogen or a methyl group;
z and y are integers independently selected from 1 to 10, preferably from 2 to 5,
R₁₀ and R₁₁ are independently selected from the group consisting of 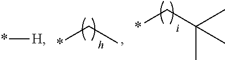
h and i are integers independently selected from 0 to 10, preferably from 1 to 5.

23. An article of manufacture incorporating the microcapsules according to any of the preceding claims.

24. The article of manufacture according to claim 23, wherein the article is selected from the group consisting of an agricultural formulation, a slurry encapsulating an agricultural active, a population of dry microcapsules encapsulating an agricultural active, an agricultural formulation encapsulating an insecticide, and an agricultural formulation for delivering a preemergent herbicide.

25. The article of manufacture according to claim 24 wherein the agricultural active is selected from the group consisting of an agricultural herbicide, an agricultural pheromone, an agricultural pesticide, an agricultural nutrient, an insect control agent and a plant stimulant.
